(19) United States
(12) Patent Application Publication
(10) Pub. No.: US 2026/0321475 A1
(43) Pub. Date: Oct. 15, 2026

(54) TEMPORALLY FOCUSED MAGNET SYSTEM AND METHODS OF USE THEREOF

(71) Applicant: UNANDUP, LLC, St. Louis, MO (US)

(72) Inventor: Francis M. CREIGHTON, St. Louis, MO (US)

(73) Assignee: UNANDUP, LLC, St. Louis, MO (US)

(21) Appl. No.: 19/683,125

(22) Filed: May 20, 2026

Related U.S. Application Data
(63) Continuation of application No. PCT/US2024/056523, filed on Nov. 19, 2024.
(60) Provisional application No. 63/600,811, filed on Nov. 20, 2023.

(51) Int. Cl. H01F 7/02; H02K 7/00

(52) U.S. Cl. CPC H01F 7/0273; H02K 7/00

(57) ABSTRACT

Disclosed herein are systems and methods for controlling magnetically controllable objects. The system can include a magnet assembly, a motor, and a motor spindle. The magnet assembly can include at least two magnets. Each magnetic can have a magnetization direction. The system is configured to move the magnetic either towards or away from the system along a central axis of motion.

### CROSS-REFERENCE TO RELATED APPLICATIONS

This application is a continuation of International Application No. PCT/US2024/056523, filed on Nov. 19, 2024, which claims priority to and the benefit of U.S. Provisional Patent Application No. 63/600,811, filed Nov. 20, 2023, the entire contents of which are incorporated herein by reference in their entirety.

### GOVERNMENT SUPPORT

This invention was made with government support under 1R44NS122604 awarded by the National Institute of Health. The government has certain rights in the invention.

### FIELD OF DISCLOSURE

The present disclosure relates to a temporally focused magnet system and methods of use thereof. The temporally focused magnet system can include a magnet assembly, a motor, a motor spindle, and a mounting mechanism configured to mount the magnet assembly to the motor spindle.

### BACKGROUND

In recent years, magnetically controllable objects have emerged as important players in modern medicine, with applications ranging from contrast agents in medical imaging to carriers for gene delivery into individual cells. Magnetically controllable objects have many beneficial therapeutic uses including improved therapeutic delivery of thrombolytic agents to clots associated with acute ischemic stroke, and treatment of ST-elevated myocardial infarctions, pulmonary embolisms, deep vein thrombosis, peripheral arterial disease, and occluded vascular access ports. However, many magnetic systems for controlling these objects have drawbacks. Prior magnet systems use magnets that are not arranged nor moved in time to more efficiently convey magnetically controllable objects within a broad region, thereby resulting in less effective and more complicated systems.

Therefore, there is a need for a temporally focused magnet system for controlling magnetically controllable objects.

### SUMMARY

Provided herein is a magnet system for controlling movement of one or more magnetically controllable objects. The system can include a magnet assembly, a motor, a motor spindle, and a mounting mechanism. The magnet assembly can include at least two magnets. Each magnet can have a magnetization direction. In an aspect, the motor can be coupled to the motor spindle. In an aspect, the mounting mechanism can be configured to mount the magnet assembly to the motor spindle. In an aspect, the magnet assembly is configured to rotate along a center axis defined by the motor spindle. In an aspect, the at least two magnets include a first magnet, a second magnet, and a third magnet. In an aspect, the magnet assembly has a center of mass aligned with the center axis to ensure stable rotation about the center axis.

In various aspects, the magnet assembly generates a changing magnetic field in time which induces at least a portion of the one or more magnetically controllable objects to move towards a preferred point or region in space by generating a time-averaged magnetic force in a general direction of desired travel towards the preferred point or region. In another aspect, after a period of time the one or more magnetically controllable objects move in a general direction of desired travel towards the preferred point or region. In an aspect, the magnet assembly generates a changing magnetic field in time which induces at least a portion of the one or more magnetically controllable objects to move away from a preferred point or region in space by generating a time-averaged magnetic force in a general direction of desired travel away from the preferred point or region. In an aspect, after a period of time the one or more magnetically controllable objects all move in the general direction of desired travel away from the preferred point or region.

In various aspects, the one or more magnetically controllable objects are permeable magnetic objects that are magnetically saturated or unsaturated, electrically conductive materials, permanent magnets, or any combination thereof. In an aspect, a time-averaged force in a general direction of desired magnetically controllable object conveyance for saturated magnetic objects is increased by orienting the magnetization directions of the at least two magnets in the magnet assembly such that a magnitude of a time average of a first order spatial gradient of a magnetic field in the general direction of desired magnetically controllable object conveyance is maximized. In another aspect, a time-averaged force in a general direction of desired magnetically controllable object conveyance for unsaturated magnetic objects is increased by orienting the magnetization directions of the at least two magnets in the magnet assembly such that a magnitude of a time average of a product of a magnetic field and a first order spatial gradient of the magnetic field in the general direction of desired magnetically controllable object conveyance is maximized.

In various aspects, the system further includes a cover configured to enclose the system. In another aspect, the system further includes one or more attachment mechanisms for attaching the cover to the system. In an aspect, the at least two magnets of the magnet assembly comprise a distribution of permanent magnetic materials. In an aspect, the magnet assembly is rotated by the motor spindle at a constant rate or a variable rate. In an aspect, the constant rate or the variable rate is reversed in time. In an aspect, the one or more magnetically controllable objects are selected from the group consisting of magnetic particulates, magnetic nanoparticles, magnetic beads, magnetic spheres, magnetic cylinders, magnetic cubes, magnetic fluids, magnetic therapeutics, iron oxide nanoparticles, magnetic contrast agents, magnetic particles used to adjunctively convey therapeutic agents creating artificial fluidic currents within a vasculature, and magnetic nanoparticles having therapeutic agents attached. In an aspect, the magnet assembly is configured to only rotate about the center axis.

Further provided herein is a method for conveying magnetically controllable objects. The method can include providing a magnet system, orienting the magnetization direction of each of at least two magnets in the magnet assembly to increase a time-average force in a general direction of desired magnetically controllable object conveyance, introducing a plurality of magnetically controllable objects into a subject, and conveying the magnetically controllable objects in the general direction of desired magnetically controllable object conveyance using the magnet assembly. In an aspect, the magnet assembly can include at least two magnets, a motor coupled to a motor spindle, and a mounting mechanism configured to mount the magnet assembly to the motor spindle. In an aspect, each magnet of the magnet assembly has a magnetization direction. In an aspect, the time-average force acting on one or more magnetically controllable objects is increased in saturated magnetic objects by orienting the magnetization directions of the at least two magnets such that a magnitude of a first order spatial gradient of a magnetic field is maximized. In another aspect, the time-average force is increased in unsaturated magnetic objects by orienting the magnetization directions of the at least two magnets such that a magnitude of a time average of a product of a magnetic field and a first order spatial gradient of the magnetic field is maximized.

Other aspects and iterations of the invention are described more thoroughly below.

### BRIEF DESCRIPTION OF FIGURES

The description will be more fully understood with reference to the following figures and graphs, which are presented as various embodiments of the disclosure and should not be construed as a complete recitation of the scope of the disclosure. It is noted that, for purposes of illustrative clarity, certain elements in various drawings may not be drawn to scale. Understanding that these drawings depict only exemplary embodiments of the disclosure and are not therefore to be considered limiting of its scope, the principles herein are described and explained with additional specificity and detail through the use of the accompanying drawings in which:

FIG. 1 is an exploded view of a magnet system in one example.

FIG. 2 is an assembled view of a magnet system in one example.

FIG. 3 is a front view of a magnet system in one example.

FIG. 4 is cross section view of a magnet system corresponding to cross section A-A of FIG. 3.

FIG. 5 is an exploded view of a magnet system in one example.

FIG. 6 is an assembled view of a magnet system in one example.

FIG. 7 is a front view of a magnet system in one example.

FIG. 8 is a cross section view of a magnet system corresponding to cross section A-A of FIG. 7.

FIG. 9 is a diagram of magnetic parameters of a magnet system in one example.

FIG. 10A is a side view of a magnet system in one example.

FIG. 10B is a front view of a magnet system in one example.

FIG. 11 is a graph of a static magnetic field generated by one magnet system in one example.

FIG. 12 is a graph of static magnetic fields generated by the magnet system in one example.

FIG. 13 is a graph of static magnetic fields generated by the magnet system in one example.

FIG. 14 is a static magnetic field plot generated by the magnet system in the x-y plane (z=0) for the magnet system.

FIG. 15 is a static magnetic field plot generated by the magnet system in the y-z plane (x=0) for the magnet system.

FIG. 16 is a static magnetic field plot generated by the magnet system in the x-z plane (y=0) for the magnet system.

FIG. 17 is a plot of the time-average magnetic field in the x-y plane (z=0) generated by the magnet system.

FIG. 18 is a plot of the time-average magnetic field in the x-y plane (z=0) generated by the magnet system.

FIG. 19 is a plot of the time-average magnetic field in the x-y plane (z=0) generated by the magnet system.

FIG. 20 is a plot of the time-average magnetic radial force metric in the x-y plane generated by the magnet system for unsaturated magnetic nanoparticles.

FIG. 21 is a plot of the time-average magnetic radial force metric in the x-y plane generated by the magnet system for saturated magnetic nanoparticles.

FIG. 22 is a graph of a radial location that maximizes the time-average radial force metric towards the magnetic system's rotation axis for unsaturated magnetic nanoparticles.

FIG. 23 is a graph of a radial location that maximizes the time-average radial force metric towards the magnetic system's rotation axis for saturated magnetic nanoparticles.

FIG. 24 is a graph of a static magnetic field comparison along the x-axis of the theoretical magnetic field compared to the measured magnetic field.

FIG. 25 is a graph of a static magnetic field comparison along the x-axis of the theoretical magnetic field compared to the measured magnetic field.

FIG. 26 is a graph of the magnetic field differential between theoretical and measured magnetic fields generated by the magnetic system.

FIG. 27 is a graph showing a change in the standard deviation for static magnetic fields in the y direction along the x-axis generated by the magnetic system.

FIG. 28 is a graph of the deviation generated by the manufactured magnet's static magnetic field from the y-direction along the x-axis.

FIG. 29 illustrates magnet system placement for maximal effect on magnetic objects.

FIG. 30 is a flow chart of a method in one example.

FIG. 31 is a diagram illustrating an example of a system for implementing certain aspects of the present technology.

Reference characters indicate corresponding elements among the views of the drawings. The headings used in the figures do not limit the scope of the claims.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and such references mean at least one of the embodiments.

Reference to “one embodiment”, “an embodiment”, or “an aspect” means that a particular feature, structure, or characteristic described in connection with the embodiment is included in at least one embodiment of the disclosure. The appearances of the phrase “in one embodiment” or “in one aspect” in various places in the specification are not necessarily all referring to the same embodiment, nor are separate or alternative embodiments mutually exclusive of other embodiments. Moreover, various features are described which may be exhibited by some embodiments and not by others.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any example term. Likewise, the disclosure is not limited to various embodiments given in this specification.

As used herein, “about” refers to numeric values, including whole numbers, fractions, percentages, etc., whether or not explicitly indicated. The term “about” generally refers to a range of numerical values, for instance, ±0.5-1%, ±1-5% or ±5-10% of the recited value, that one would consider equivalent to the recited value, for example, having the same function or result.

The term “magnetically controllable object” includes objects that are magnetically sensitive or magnetic objects. For example, magnetically controllable objects can include saturated permeable magnetic objects, unsaturated permeable magnetic objects, permanent magnets, electrically conductive materials, and other magnetically controllable materials.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims or can be learned by the practice of the principles set forth herein.

Provided herein is a temporally focused magnet system for controlling magnetically controllable objects. The system can include a magnet assembly, a motor coupled to a motor spindle, and a mounting mechanism. The magnet assembly can include at least two magnets, and each magnet can have a magnetization direction. The mounting mechanism can be configured to mount the magnet assembly to the motor spindle. In an example, the magnet assembly is configured to rotate along a center axis defined by the motor spindle. The system can be configured to control movement of one or more magnetically controllable objects. For example, the rotation of the magnet assembly around a single axis of rotation can provide a more compact system that generates a changing magnetic field in time which can induce movement of the one or more magnetically controllable objects. Existing magnet systems are typically large and not easily movable, such that they remain in the same room and require patients to come to the system. In the present magnet system, the arrangement of the magnets such that they can be rotated around a single axis allows the system to be more compact and portable. This would allow a hospital to have and store multiple systems that can be moved to the patient. Further, the magnet system maintains stability by only rotating around a single axis and reduces the complexity of the magnetization directions of the magnets needed to maximize time-averaged forces on the one or more magnetically controllable objects in a desired direction. The area over which the one or more magnetically controllable objects are conveyed (e.g., toward or away from the center axis of the magnetic system) is broad, which reduces the impact on efficacy with respect to precise magnet system placement. The magnet system can be optimized to provide a maximum time-averaged magnetic force at a particular point in space.

In some aspects, the magnet system can be small enough and light enough (in weight) such that it can travel with transferred patients. In some examples, the magnet system can have a total length of about 150 mm to about 160 mm, about 160 mm to about 170 mm, about 170 mm to about 180 mm, about 180 mm to about 190 mm, about 190 mm to about 200 mm, about 200 mm to about 210 mm, about 220 mm to about 230 mm, about 230 mm to about 240 mm, about 240 mm to about 250 mm, about 250 mm to about 300 mm, or more. The magnet system can have a total height of about 150 mm to about 160 mm, about 160 mm to about 170 mm, about 170 mm to about 180 mm, about 180 mm to about 190 mm, about 190 mm to about 200 mm, about 200 mm to about 210 mm, about 220 mm to about 230 mm, about 230 mm to about 240 mm, about 240 mm to about 250 mm, about 250 mm to about 300 mm, or more. The magnet system can have a total width of 150 mm to about 160 mm, about 160 mm to about 170 mm, about 170 mm to about 180 mm, about 180 mm to about 190 mm, about 190 mm to about 200 mm, about 200 mm to about 210 mm, about 220 mm to about 230 mm, about 230 mm to about 240 mm, about 240 mm to about 250 mm, about 250 mm to about 300 mm, or more. The magnet system can have a total weight of about 3 kg to about 4 kg, about 4 kg to about 5 kg, about 5 kg to about 6 kg, about 6 kg to about 7 kg, about 7 kg to about 8 kg, about 8 kg to about 9 kg, about 9 kg to about 10 kg, or more. In some examples, each magnet in the magnet system can have a weight of about 2 kg to about 3 kg, about 3 kg to about 4 kg, about 4 kg to about 5 kg, about 5 kg to about 6 kg, about 6 kg to about 7 kg, about 7 kg to about 8 kg, about 8 kg to about 9 kg, about 9 kg to about 10 kg, about 10 kg to about 11 kg, or more. In some examples, when the magnet has a greater weight it produces a greater magnetic field and thereby a greater magnetic force.

In some aspects, magnetically controllable objects to be controlled by the system can include magnetic particulates, magnetic nanoparticles, magnetic beads, magnetic spheres, magnetic cylinders, magnetic cubes, magnetic fluids, magnetic therapeutics, iron oxide nanoparticles, and magnetic contrast agents. Magnetically controllable objects can include magnetic nanoparticles to which therapeutics are attached. Magnetically controllable objects can include magnetic nanoparticles used to adjunctively convey therapeutic agents by creating artificial fluidic currents within a vasculature of a patient. Therapeutic agents which can be attached to magnetic nanoparticles, or be adjunctively conveyed by magnetic nanoparticles, include, but are not limited to, therapeutic agents used to treat cancer or provide neuroprotection. Magnetically controllable objects can also include permanent magnetic materials, magnetically saturated permeable magnetic materials, magnetically unsaturated permeable magnetic materials, electrically conductive materials, and other magnetically controllable or magnetically sensitive materials. Magnetically controllable objects can also include radiodense magnetic objects.

The systems and methods described herein can be used for therapeutic uses in some examples. For example, therapeutic uses can include improved therapeutic delivery of thrombolytic agents to clots associated with acute ischemic stroke, treatment of ST-elevated myocardial infarctions, pulmonary embolisms, deep vein thrombosis, peripheral arterial disease, and occluded vascular access ports. Other therapeutic uses can include control of magnetic embolization beads (or other particulates) to embolize tumors. Additional therapeutic uses can include magnetically controllable objects to deliver hypothermia, which can destroy selected tissues (e.g., tumors). Magnetically controllable objects can include magnetic objects that are radiodense which can be used as contrast to improve vessel, organ, and other biological imaging. The systems and methods described herein can also be used for non-therapeutic uses such as in engineering or manufacturing where the controlled conveyance of magnetically controllable objects is desired. Further the systems and methods described herein can be used in microreactors in energy systems.

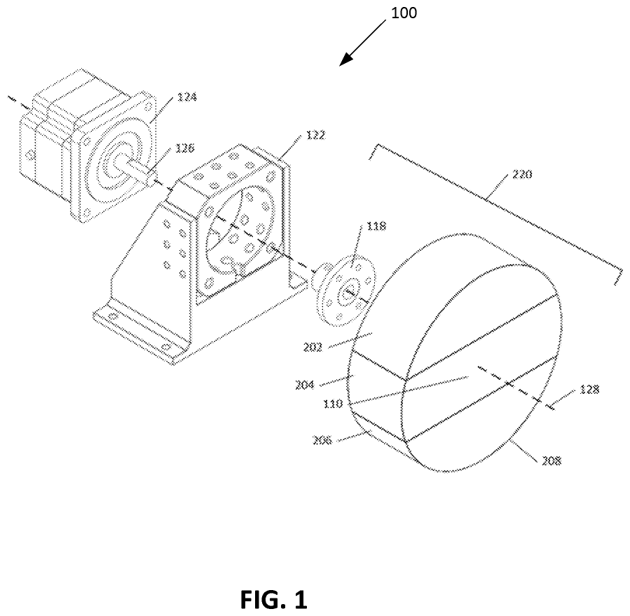

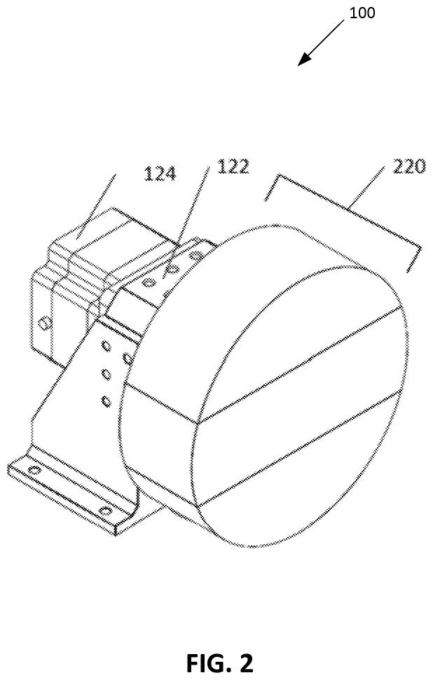

FIGS. 1-4 illustrate an example magnet system 100. The magnet system 100 can include a magnet assembly 220. The magnet assembly 220 includes at least two magnets. For example, the magnet assembly 220 can include, but not limited to, 2, 3, 4, 5, 6, 7, 8, 9, 10 or more magnets. In some examples, a large number of magnets can be used which can be configured in optimal orientations to improve the action of temporal magnetic fields. In some embodiments, the magnet assembly 220 can include a first magnet 202, a second magnet 204, and a third magnet 206. In an example, the first magnet 202, second magnet 204, and third magnet 206 can be fixed to each other. In some examples, when the first magnet 202, second magnet 204, and third magnet 206 are fixed to each other they can form a circular magnet assembly 220. The first magnet 202, second magnet 204, and third magnet 206 can each be a single magnet or each can be composed of a distribution of multiple individual permanent magnets.

In some examples, the first magnet 202, the second magnet 204, and the third magnet 206 can be Neodymium Boron Iron. In other examples, the first magnet 202, second magnet 204, and third magnet 206 can be samarium cobalt, platinum cobalt, platinum iron, bonded magnets, electromagnets, or any magnet operable to maintain a permanent magnetization.

The magnet assembly 220 can further include a mounting mechanism. In some examples, the mounting mechanism can be a mounting flange 118. The mounting flange 118 can be configured to couple to the first magnet 202, second magnet 204, and/or third magnet 206. In some examples, the mounting flange can permanently couple to the first magnet 202, the second magnet 204, and/or the third magnet 206. In other examples, the mounting flange 118 can removably couple to the first magnet 202, the second magnet 204, and/or the third magnet 206. The mounting flange 118 can be removably coupled to the first magnet 202, the second magnet 204, and the third magnet 206 using screws, snap-fit connectors, or other connection mechanisms known in the art.

The magnet system 100 can further include a motor 124 and a motor spindle 126. The motor 124 can be connected to a power source via a power supply cable or have a batter operable to provide power to the motor 124. In an example, the motor 124 can be a stepper motor or other motor known in the art to provide rotation to the motor spindle 126. The motor spindle 126 can be configured to couple to the mounting flange 118. The motor 124 is operable to provide a torque to the motor spindle 126, thereby rotating the magnet assembly 220. The magnet assembly 220 can rotate about the center axis 128 which is defined by the motor spindle 126. The motor 124 can provide a rotation rate and rotation direction (e.g., clockwise or counterclockwise) to the magnet assembly 220. In an example, the rotation rate and rotation direction can be dynamically changed. In another example, the rotation rate and rotation direction can be optimized to maximize the time-average force acting on one or more magnetically controllable objects.

The motor 124 can supply a constant rate of rotation or a variable rate of rotation. In an example, the constant rate can be about 1 Hz to about 2 Hz, about 2 Hz to about 3 Hz, about 3 Hz to about 4 Hz, about 4 Hz to about 5 Hz, about 5 Hz to about 6 Hz, about 6 Hz to about 7 Hz, about 7 Hz to about 8 Hz, about 8 Hz to about 9 Hz, about 9 Hz to about 10 Hz, or more. Variable rates can include rates between about 1 Hz to about 1 kHz. In an example, variable rates can include varying rates between about 1 Hz to about 5 Hz, about 5 Hz to about 10 Hz, about 3 Hz to about 10 Hz, about 1 Hz to about 10 Hz, or any range therebetween. Rates of rotation can include variable and/or constant rates that do not generate substantial electromagnet interference (EMI). In some examples, the constant or variable rate of rotation is any rate of rotation under about 1 kHz. In some examples, rates of rotation of about 3 Hz to about 10 Hz can be used which can be filtered from ECG signals, do not generate EMI, and do not risk traumatic mechanical failure if the magnet system fails during use.

In an example, the constant rate or the variable rate can be reversed in time. Reversing the rate of rotation of the motor 124 can cause the magnetically controllable objects to climb the opposite wall to the target location (e.g., opposite wall of a vessel). By reversing the rate, the magnetically controllable objects can be better conveyed to the relevant biology (e.g., occluded vessels, tumors, etc.).

The magnet assembly 220 can have a center of mass aligned with the center axis 128 to ensure stable rotation about the center axis 128. In some examples, the magnet assembly 220 only rotates about one axis (i.e., the center axis 128). In an example, the second magnet 204 is not offset from the center axis 128, thereby minimizing the moment of inertia about the center axis 128. By rotating the magnet assembly 220 about only one axis, the magnet system 100 is cheaper to manufacture, safer because the magnet assembly 220 is rotated about the center of mass of the magnet assembly 220, easier to remove and replace a magnet assembly 220 on the motor spindle 126, and it is easier to measure speed of magnet assembly 220 rotation and the exact placement. In some examples, the exact placement of the magnet system can be determined using an encoder on the motor spindle 126.

The magnet system can further include a base 122. The base 122 can be operable to stabilize the magnet system on a surface. In some examples, the base 122 can have screw holes operable to receive screws to secure the base 122 to a surface. In other examples, other securement mechanisms known in the art can be used to secure the base 122 to a surface.

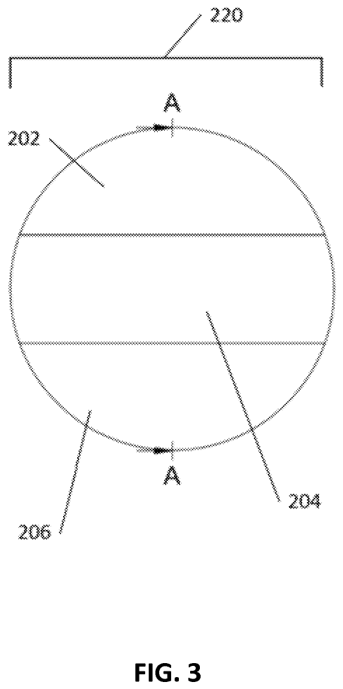

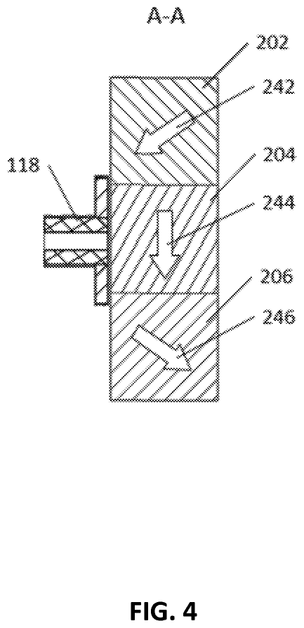

FIG. 4 illustrates a cross section A-A of the magnet assembly 220. The cross section line A-A is illustrated on FIG. 3. As illustrated, the first magnet 202 can have a magnetization direction 242, the second magnet 204 can have a magnetization direction 244, and the third magnet 206 can have a magnetization direction 246. The magnetization direction 242 of the first magnet 202, the magnetization direction 244 of the second magnet 204, and the magnetization direction 246 of the third magnet 206 can be oriented to increase the time-average force generated on the magnetically controlled objects such that magnetically controlled object movement occurs in a desired direction. Preferred magnetization directions magnetization directions 242, 244, 246 can be used to maximize the generated time-average force acting on one or more magnetically controllable objects.

In some aspects, the magnetization directions 242, 244, 246 depend on the magnetically controllable objects to be controlled. The magnetically controllable objects can be permanent magnets, permeable magnetic objects, electrically conductive materials, or other magnetically controllable objects. The magnetization directions 242, 244, 246 can be oriented such that, when rotated by the motor 124 and motor spindle 126, the magnet assembly 220 generates a changing magnetic field in time which induces at least a portion of the one or more magnetically controllable objects to move towards a preferred point or region in space by generating a time-averaged magnetic force in a general direction of desired travel towards the preferred point or region. The term “general” means that most of the magnetically controllable objects move in the direction of desired travel, while some magnetically controllable objects can move slightly off track from the direction of desired travel before correcting after a period of time. In some examples, after a period of time all of the one or more magnetically controllable objects move in the general direction of desired travel towards the preferred point or region. In some examples, the period of time can depend on the biology where the magnetically controllable objects are being conveyed, the type of magnetically controllable objects, the magnetic forces generated, and magnet system's temporal field. In an example, magnetic nanoparticles can move at about 10 mm/min. For some therapies, the period of time can be about 15 minutes to about 20 minutes, about 20 minutes to about 25 minutes, about 25 minutes to about 30 minutes, about 30 minutes to about 35 minutes, about 35 minutes to about 40 minutes, about 40 minutes to about 45 minutes, about 45 minutes to about 50 minutes, about 50 minutes to about 55 minutes, about 55 minutes to about 60 minutes, or more.

In other examples, the magnetization directions 242, 244, 246 can be oriented such that, when rotated by the motor 124 and motor spindle 126, the magnet assembly 220 generates a changing magnetic field in time which induces at least a portion of the one or more magnetically controllable objects to move away from a preferred point or region in space by generating a time-averaged magnetic force in a general direction of desired travel away from the preferred point or region. In an example, after a period of time, the one or more magnetically controllable objects all move in the general direction of desired travel away from the preferred point or region. In some examples, the period of time can depend on the biology where the magnetically controllable objects are being conveyed, the type of magnetically controllable objects, the magnetic forces generated, and magnet system's temporal field. In an example, magnetic nanoparticles can move at about 10 mm/min. For some therapies, the period of time can be about 15 minutes to about 20 minutes, about 20 minutes to about 25 minutes, about 25 minutes to about 30 minutes, about 30 minutes to about 35 minutes, about 35 minutes to about 40 minutes, about 40 minutes to about 45 minutes, about 45 minutes to about 50 minutes, about 50 minutes to about 55 minutes, about 55 minutes to about 60 minutes, or more.

In some aspects, the time-averaged force in a general direction of desired magnetically controllable object conveyance for saturated permeable magnetic objects and permanent magnetic objects is increased by orienting the magnetization directions 242, 244, 246 such that a magnitude of a time average of a first order spatial gradient of a magnetic field in the general direction of desired magnetically controllable object conveyance is maximized. In another example, a time-averaged force of desired magnetically controllable object conveyance for unsaturated magnetic objects is increased by orienting the magnetizations direction 242, 244, 246 such that a magnitude of a time average of a product of a magnetic field and a first order spatial gradient of the magnetic field in the general direction of desired magnetically controllable object conveyance is maximized. In some examples, the magnetization directions 242, 244, 246 are different depending on the type of magnetically controllable objects to be conveyed (e.g., saturated, unsaturated, permanent, electrically conductive materials, etc.). For unsaturated permeable magnetic objects, the force is proportional to the applied magnetic field and magnetic gradient of the magnet system 100. For saturated permeable magnetic objects and permanent magnetic objects, the magnetic force is proportional only to the magnetic gradient of the magnet system 100.

The area over which the one or more magnetically controllable objects are conveyed by the magnet system 100 is broad, which reduces the impact on efficacy with respect to precise magnet system placement. In an aspect, for magnetically saturated magnetic objects and permanent magnetic objects, the magnetization directions 242, 244, 246 to maximize time average forces in the direction towards the center axis 128 at a point located about 13 cm in from the magnet assembly 220 front surface 208 and about 11 cm in the radial direction from the center axis 128 can be about 29 degrees for magnetization direction 246, about 90 degrees for magnetization direction 244, and about 151 degrees for magnetization direction 242. In another aspect, for magnetically saturated magnetic objects and permanent magnetic objects, the magnetization directions 242, 244, 246 to maximize time average forces in the direction towards the center axis 128 at a point located about 5 cm to about 20 cm, about 5 cm to about 10 cm, about 10 cm to about 15 cm, or about 15 cm to about 20 cm in from the magnet assembly 220 front surface 208 and about 5 cm to about 20 cm, about 5 cm to about 10 cm, about 10 cm to about 15 cm, or about 15 cm to about 20 cm in the radial direction from the center axis 128 can be about 25 degrees to about 35 degrees, about 25 degrees to about 27 degrees, about 27 degrees to about 30 degrees, about 30 degrees to about 32 degrees, or about 32 degrees to about 35 degrees for magnetization direction 246, about 85 degrees to about 95 degrees, about 85 degrees to about 87 degrees, about 87 degrees to about 90 degrees, about 90 degrees to about 92 degrees, or about 92 degrees to about 95 degrees for magnetization direction 244, and about 145 to about 155 degrees, about 145 degrees to about 147 degrees, about 147 degrees to about 150 degrees, about 150 degrees to about 152 degrees, or about 152 degrees to about 155 degrees for magnetization direction 242.

In another aspect, for magnetically saturated magnetic objects and permanent magnetic objects, the magnetization directions 242, 244, 246 to maximize time average forces in the direction towards the center axis 128 at a point located about 5 cm to about 50 cm, about 5 cm to about 10 cm, about 10 cm to about 15 cm, about 15 cm to about 20 cm, about 25 cm to about 30 cm, about 30 cm to about 35 cm, about 35 cm to about 40 cm, about 40 cm to about 45 cm, about 45 cm to about 50 cm, or more in from the magnet assembly 220 front surface 208 and about 5 cm to about 20 cm, about 5 cm to about 10 cm, about 10 cm to about 15 cm, or about 15 cm to about 20 cm in the radial direction from the center axis 128 can be about 20 degrees to about 40 degrees, about 20 degrees to about 22 degrees, about 22 degrees to about 25 degrees, about 25 degrees to about 27 degrees, about 27 degrees to about 30 degrees, about 30 degrees to about 32 degrees, about 32 degrees to about 35 degrees, about 35 degrees to about 37 degrees, or about 37 degrees to about 40 degrees for magnetization direction 246, about 80 degrees to about 100 degrees, about 80 degrees to about 82 degrees, about 82 degrees to about 85 degrees, about 85 degrees to about 87 degrees, about 87 degrees to about 90 degrees, about 90 degrees to about 92 degrees, about 92 degrees to about 95 degrees, about 95 degrees to about 97 degrees, or about 97 degrees to about 100 degrees for magnetization direction 244, and about 140 to about 160 degrees, about 140 degrees to about 142 degrees, about 142 degrees to about 145 degrees, about 145 degrees to about 147 degrees, about 147 degrees to about 150 degrees, about 150 degrees to about 152 degrees, about 152 degrees to about 155 degrees, about 155 degrees to about 157 degrees, or about 157 degrees to about 160 degrees for magnetization direction 242.

In another aspect, for magnetically unsaturated magnetic objects, the magnetization directions 242, 244, 246 to maximize time average forces in the direction towards the center axis 128 at a point located about 13 cm in from the magnet assembly 220 front surface 208 and about 11 cm in the radial direction from the center axis 128 can be about 38 degrees for magnetization direction 246, about 90 degrees for magnetization direction 244, and about 142 degrees for magnetization direction 242. In another aspect, for magnetically unsaturated magnetic objects, the magnetization directions 242, 244, 246 to maximize time average forces in the direction towards the center axis 128 at a point located about 5 cm to about 20 cm, about 5 cm to about 10 cm, about 10 cm to about 15 cm, or about 15 cm to about 20 cm in from the magnet assembly 220 front surface 208 and about 5 cm to about 20 cm, about 5 cm to about 10 cm, about 10 cm to about 15 cm, or about 15 cm to about 20 cm in the radial direction from the center axis 128 can be about 30 to about 45 degrees, about 30 degrees to about 30 degrees to about 35 degrees, about 35 degrees to about 40 degrees, or about 40 degrees to about 45 degrees for magnetization direction 246, about 85 degrees to about 95 degrees, about 85 degrees to about 90 degrees, or about 90 degrees to about 95 degrees for magnetization direction 244, and about 135 degrees to about 150 degrees, about 135 degrees to about 140 degrees, about 140 degrees to about 145 degrees, or about 145 degrees to about 150 degrees for magnetization direction 242.

In another aspect, for magnetically unsaturated magnetic objects, the magnetization directions 242, 244, 246 to maximize time average forces in the direction towards the center axis 128 at a point located about 5 cm to about 50 cm, about 5 cm to about 10 cm, about 10 cm to about 15 cm, about 15 cm to about 20 cm, about 25 cm to about 30 cm, about 30 cm to about 35 cm, about 35 cm to about 40 cm, about 40 cm to about 45 cm, about 45 cm to about 50 cm, or more in from the magnet assembly 220 front surface 208 and about 5 cm to about 20 cm, about 5 cm to about 10 cm, about 10 cm to about 15 cm, or about 15 cm to about 20 cm in the radial direction from the center axis 128 can be about 25 to about 50 degrees, about 25 degrees to about 27 degrees, about 27 degrees to about 30 degrees, about 30 degrees to about 35 degrees, about 35 degrees to about 40 degrees, or about 40 degrees to about 45 degrees, about 45 degrees to about 47 degrees, or about 47 degrees to about 50 degrees for magnetization direction 246, about 80 degrees to about 100 degrees, about 80 degrees to about 85 degrees, about 85 degrees to about 90 degrees, about 90 degrees to about 95 degrees, about 95 degrees to about 100 degrees for magnetization direction 244, and about 130 degrees to about 155 degrees, about 130 degrees to about 135 degrees, about 135 degrees to about 140 degrees, about 140 degrees to about 145 degrees, about 145 degrees to about 150 degrees, or about 150 degrees to about 155 degrees for magnetization direction 242.

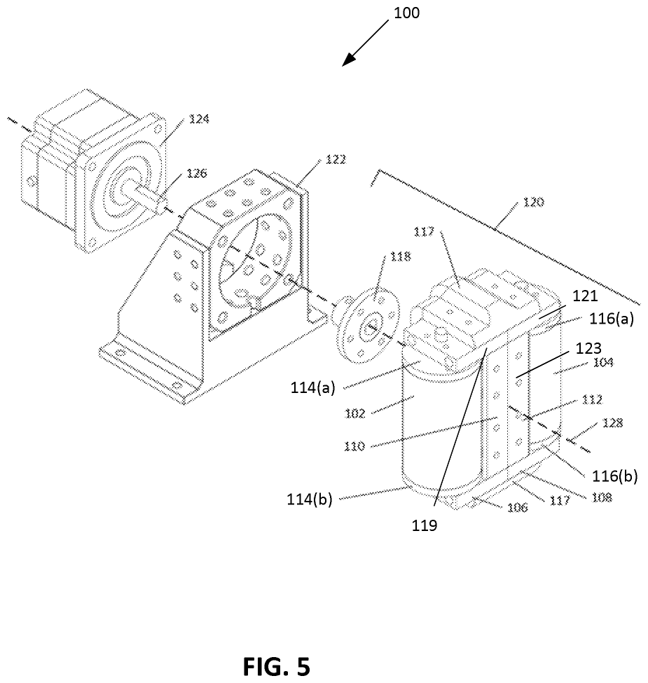

FIGS. 5-8 illustrate the magnet system 100 in another example. In this aspect, the magnet system 100 can include a magnet assembly 120, a motor 124, a motor spindle 126, and a base 122. The magnet assembly 120 can include a first magnet 102 and a second magnet 104. The first magnet 102 and second magnet 104 can be cylinders or other shapes. The first magnet 102 and the second magnet 104 can each include a distribution of individual magnets. In some examples, the first magnet 102 and second magnet 104 can be composed of a distribution of permanent magnetic materials. The distribution of magnets can share the same magnetization direction. The magnet assembly 120 can include a first lower mounting cap 106 and a second lower mounting cap 108. The first magnet 102 can have an upper end cap 114 (a) and a lower end cap 114 (b). The upper end cap 114 (a) and 114 (b) can be keyed such that the magnetic orientation of the first magnet 102 is fixed when it is inserted in the first lower mounting cap 106 and first upper mounting cap 119. The second magnet 104 can have an upper end cap 116 (a) and a lower end cap 116 (b). The upper end cap 116 (a) and the lower end cap 116 (b) can be keyed such that the magnetic orientation of the second magnet 104 is fixed when it is inserted in the second lower mounting cap 108 and the second upper mounting cap 121. Using the keys in the endcaps 114 (a), 114 (b), 116 (a), 116 (b) and the keyways in the mounting caps 106, 108, 119, 121, the magnetic orientations of the first magnet 102 and the second magnet 104 can remain in a fixed position.

In some examples, the first magnet 102 and the second magnet 104 can be Neodymium Boron Iron. In other examples, the first magnet 202, second magnet 204, and third magnet 206 can be samarium cobalt, platinum cobalt, platinum iron, bonded magnets, electromagnets, or any magnet operable to maintain a permanent magnetization.

The magnet assembly 120 can further include a center support column 123. The center support column 123 can separate the first magnet 102 and the second magnet 104. The support column 123 can be attached to a mounting yoke 117. The mounting yoke 117 includes the center support column 123 and the mounting caps 106, 108, 119, 121. In some examples, the mounting yoke 117, the center support column 123, and the mounting caps 106, 108, 119, 121 can all be manufactured as one component. In other examples, the center support column 123 and the mounting caps 106, 108, 119, and 121 can be removably coupled to the mounting yoke 117. When the center support column 123 is removably coupled to the mounting yoke 117, center support columns of different heights can be used, thereby allowing different heights of magnets to be used with the magnet system 100. Further, when the mounting caps 106, 108, 119, and 121 are removably coupled to the mounting yoke 117, the mounting caps 106, 108, 119, 121 can be coupled in different orientations thereby allowing the keyways of the mounting caps 106, 108, 119, 121 to be in different orientations. In this manner, the fixed orientation of the first magnet 102 and second magnet 104 can be adjusted by adjusting the orientation of the keyways in the mounting caps 106, 108, 119, 121, while also allowing the first magnet 102 and second magnet 104 to be in a fixed orientation when inserted in the mounting caps 106, 108, 119, 121.

The mounting yoke 117 can be configured to couple to mounting flange 118. In some examples, the mounting flange 118 can be permanently coupled to or a part of the mounting yoke 117. In other examples, the mounting flange 118 can be configured to removably couple to the mounting yoke 117. The mounting flange 118 can also be coupled to the motor spindle 126. In some examples, the mounting flange 118 can be removably coupled to the motor spindle 126. In other examples, the mounting flange 118 can be permanently coupled to or integral to the motor spindle 126.

The motor 124 is operable to provide a torque to the motor spindle 126, thereby rotating the magnet assembly 120. The magnet assembly 120 can rotate about the center axis 128 which is defined by the motor spindle 126. The motor 124 can provide a rotation rate and rotation direction (e.g., clockwise or counterclockwise) to the magnet assembly 120. In an example, the rotation rate and rotation direction can be dynamically changed. In another example, the rotation rate and rotation direction can be optimized to maximize the time-average force acting on one or more magnetically controllable objects.

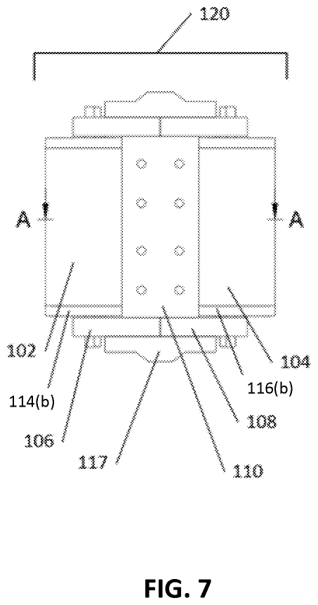

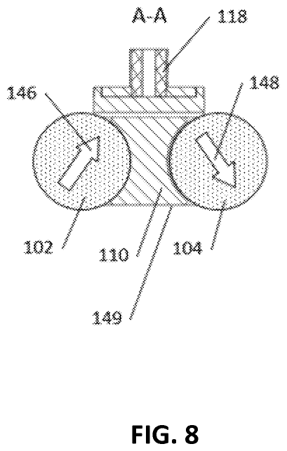

As illustrated in FIG. 8, which is a cross section of the magnet assembly 120 at line A-A in FIG. 7, the first magnet 102 can have magnetization direction 146 and the second magnet 104 can have magnetization direction 148. The magnetization directions 146, 148 can be oriented to maximize the generated time-average force acting on one or more magnetically controllable objects. In some examples, the magnetization directions 146, 148 can be changed dynamically. Magnetization directions 146, 148 can be changed dynamically through the use of one or more additional motors, including hand cranks which can turn the first magnet 102 and the second magnet 104 in unison in the same or different directions.

In some aspects, the magnetization directions 146, 148 depend on the magnetically controllable objects to be controlled. For example, the magnetically controllable objects can be permanent magnets, permeable magnetic objects, electrically conductive materials, or other magnetically controllable objects. The magnetization directions 146, 148 can be oriented such that, when rotated by the motor 124 and motor spindle 126, the magnet assembly 120 generates a changing magnetic field in time which induces at least a portion of the one or more magnetically controllable objects to move towards a preferred point in preferred point or region in space by generating a time-averaged magnetic force in a general direction of desired travel towards the preferred point or region. In some examples, after a period of time all of the one or more magnetically controllable objects move in the general direction of desired travel towards the preferred point or region. In other examples, the magnetization directions 146, 148 can be oriented such that, when rotated by the motor 124 and motor spindle 126, the magnet assembly 120 generates a changing magnetic field in time which induces at least a portion of the one or more magnetically controllable objects to move away from a preferred point or region in space by generating a time-averaged magnetic force in a general direction of desired travel away from the preferred point or region. In an example, after a period of time, the one or more magnetically controllable objects all move in the general direction of desired travel away from the preferred point or region.

In some aspects, the time-averaged force in a general direction of desired magnetically controllable object conveyance for saturated permeable magnetic objects and permanent magnetic objects is increased by orienting the magnetization directions 146, 148 such that a magnitude of a time average of a first order spatial gradient of a magnetic field in the general direction of desired magnetically controllable object conveyance is maximized. In another example, a time-averaged force of desired magnetically controllable object conveyance for unsaturated magnetic objects is increased by orienting the magnetizations direction 146, 148 such that a magnitude of a time average of a product of a magnetic field and a first order spatial gradient of the magnetic field in the general direction of desired magnetically controllable object conveyance is maximized. In some examples, the magnetization directions 242, 244, 246 are different depending on the type of magnetically controllable objects to be conveyed (e.g., saturated, unsaturated, permanent, electrically conductive materials, etc.).

The area over which the one or more magnetically controllable objects are conveyed by the magnet system 100 is broad, which reduces the impact on efficacy with respect to precise magnet system placement. In an aspect, for magnetically saturated magnetic objects and permanent magnetic objects, the magnetization directions 146, 148 to maximize time average forces in the direction towards the center axis 128 at a point located about 13 cm in from the magnet assembly 120 front surface 149 and about 2 cm in the radial direction from the center axis 128, assuming the center of the first magnet 102 is 7.5 cm from the center of the second magnet 104, are about 156 degrees from the center axis 128 for magnetization direction 146 and about 24 degrees from the center axis 128 for magnetization direction 148. In another aspect, for magnetically saturated magnetic objects and permanent magnetic objects, the magnetization directions 146, 148 to maximize time average forces in the direction towards the center axis 128 at a point located about 5 cm to about 20 cm, about 5 cm to about 10 cm, about 10 cm to about 15 cm, or about 15 cm to about 20 cm in from the magnet assembly 120 front surface 149 and about 1 cm to about 10 cm, about 1 cm to about 5 cm, or about 5 cm to about 10 cm in the radial direction from the center axis 128, assuming the center of the first magnet 102 is about 5 cm to about 10 cm from the center of the second magnet 104, are about 150 degrees to about 160 degrees, about 150 degrees to about 155 degrees, or about 155 degrees to about 160 degrees from the center axis 128 for magnetization direction 146 and about 20 degrees to about 30 degrees, about 20 degrees to about 25 degrees, or about 25 degrees to about 30 degrees for magnetization direction 148.

In another aspect, for magnetically saturated magnetic objects and permanent magnetic objects, the magnetization directions 146, 148 to maximize time average forces in the direction towards the center axis 128 at a point located about 5 cm to about 50 cm, about 5 cm to about 10 cm, about 10 cm to about 15 cm, about 15 cm to about 20 cm, about 20 cm to about 25 cm, about 25 cm to about 30 cm, about 30 cm to about 35 cm, about 35 cm to about 40 cm, about 40 cm to about 45 cm, or about 45 cm to about 50 cm in from the magnet assembly 120 front surface 149 and about 1 cm to about 20 cm, about 1 cm to about 5 cm, or about 5 cm to about 10 cm, about 10 cm to about 15 cm, or about 15 cm to about 20 cm in the radial direction from the center axis 128, assuming the center of the first magnet 102 is about 5 cm to about 10 cm from the center of the second magnet 104, are about 145 degrees to about 165 degrees, about 145 degrees to about 150 degrees, about 150 degrees to about 155 degrees, about 155 degrees to about 160 degrees, or about 160 degrees to about 165 degrees from the center axis 128 for magnetization direction 146 and about 15 degrees to about 35 degrees, about 15 degrees to about 20 degrees, about 20 degrees to about 25 degrees, about 25 degrees to about 30 degrees, or about 30 degrees to about 35 degrees for magnetization direction 148.

In another aspect, for magnetically unsaturated magnetic objects, the magnetization directions 146, 148 to maximize time average forces in the direction towards the center axis 128 at a point located about 13 cm in from the magnet assembly 120 front surface 149 and about 2 cm in the radial direction from the center axis 128, assuming the center of the first magnet 102 is 7.5 cm from the center of the second magnet 104, are about 143 degrees from the center axis 128 for magnetization direction 146 and about 37 degrees from the center axis 128 for magnetization direction 148. In another aspect, for magnetically unsaturated magnetic objects, the magnetization directions 146, 148 to maximize time average forces in the direction towards the center axis 128 at a point located about 5 cm to about 20 cm, about 5 cm to about 10 cm, about 10 cm to about 15 cm, or about 15 cm to about 20 cm in from the magnet assembly 120 front surface 149 and about 1 cm to about 10 cm, about 1 cm to about 5 cm, or about 5 cm to about 10 cm in the radial direction from the center axis 128, assuming the center of the first magnet 102 is about 5 cm to about 10 cm from the center of the second magnet 104, are about 140 degrees to about 150 degrees, about 140 degrees to about 145 degrees, or about 145 degrees to about 150 degrees from the center axis 128 for magnetization direction 146 and about 30 degrees to about 40 degrees, about 30 degrees to about 35 degrees, or about 35 degrees to about 40 degrees from the center axis 128 for magnetization direction 148.

In another aspect, for magnetically unsaturated magnetic objects, the magnetization directions 146, 148 to maximize time average forces in the direction towards the center axis 128 at a point located about 5 cm to about 50 cm, about 5 cm to about 10 cm, about 10 cm to about 15 cm, about 15 cm to about 20 cm, about 20 cm to about 25 cm, about 25 cm to about 30 cm, about 30 cm to about 35 cm, about 35 cm to about 40 cm, about 40 cm to about 45 cm, or about 45 cm to about 50 cm in from the magnet assembly 120 front surface 149 and about 1 cm to about 20 cm, about 1 cm to about 5 cm, about 5 cm to about 10 cm, about 10 cm to about 15 cm, or about 15 cm to about 20 cm in the radial direction from the center axis 128, assuming the center of the first magnet 102 is about 5 cm to about 10 cm from the center of the second magnet 104, are about 135 degrees to about 155 degrees, about 135 degrees to about 140 degrees, about 140 degrees to about 145 degrees, about 145 degrees to about 150 degrees, or about 150 degrees to about 155 degrees from the center axis 128 for magnetization direction 146 and about 25 degrees to about 45 degrees, about 25 degrees to about 30 degrees, about 30 degrees to about 35 degrees, about 35 degrees to about 40 degrees, or about 40 degrees to about 45 degrees from the center axis 128 for magnetization direction 148.

In some aspects, the magnet systems described herein can further include a cover. The cover can be operable to cover the entire magnet system to fully enclose the magnetic system. In some examples, a cover may be required to prevent any risks of an uncovered rotating magnet assembly. In some examples, the magnet system 100 can include one or more attachment mechanisms for attaching the cover to the magnet system. For example, the one or more attachment mechanisms can be snap-fit connectors, latches, Velcro, or other attachment mechanisms known in the art. In an example, the cover can include one or more shrouds configured to fully enclose the magnet system 100. Risks prevented by use of the cover can include reducing or preventing bump, pinch, and twist forces that could be injurious if in contact with a subject. The cover incapsulates the magnet system 100 in case of mechanical failure. The cover prevents items from being pulled into the magnet assembly of the magnet system 100. The cover also prevents non-magnetic objects from being dropped into the mechanics of the magnet system 100. The cover further ensures that loose items are not catapulted from the magnet assembly when the magnet assembly is rotating.

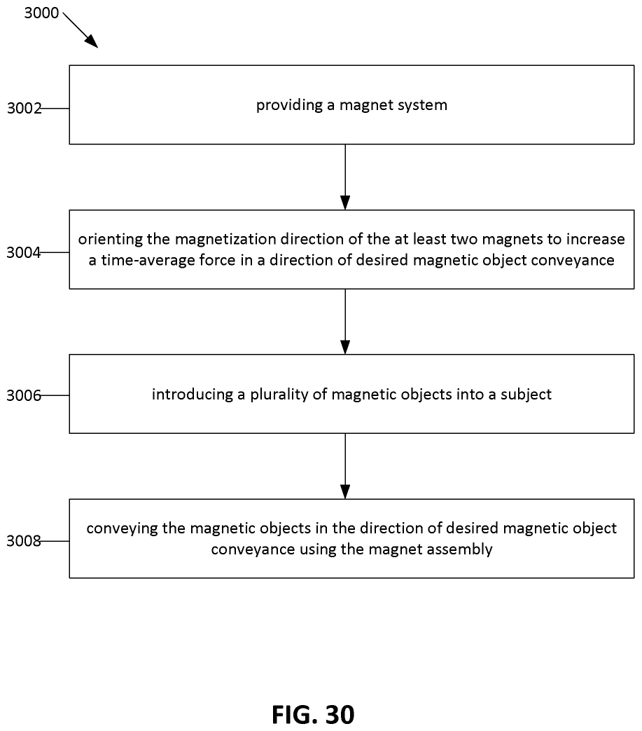

Further provided herein is a method for controlling magnetically controllable objects. FIG. 30 illustrates the method 3000 for controlling magnetically controllable objects in one example. The method 3000 can begin at block 3002. At block 3002, the method 3000 can include providing a magnet system. The magnetic system can be any of the magnet systems described herein. In some examples, the magnet system can include a magnet assembly, a motor coupled to a motor spindle, and a mounting mechanism configured to mount the magnet assembly to the motor system. In some examples, the magnet assembly can include at least two magnets. Each magnet can have a magnetization direction. In some examples, the magnet assembly can include two, three, four, five, six, seven, eight, nine, ten or more magnets where each magnet has a magnetization direction.

At block 3004, the method 3000 can include orienting the magnetization direction of each of the at least two magnets to increase a time-average force in a general direction of desired magnetically controllable object conveyance. In some examples, orienting the magnetization direction can depend on the type of magnetically controllable objects to be controlled. For example, the magnetically controllable objects can be permanent magnets, permeable magnetic objects, electrically conductive materials, or other magnetically controllable objects. In some examples, the magnetization directions can be the same for permanent magnets and saturated permeable magnets. In an example, the time-average force for magnetically saturated magnetic objects is increased by orienting the magnetization directions of the magnets such that a magnitude of a first order spatial gradient of the magnetic field is maximized. For magnetically unsaturated magnetic objects, the time-average force is increased by orienting the magnetization direction such that a magnitude of a time-average product of a magnetic field and a first order spatial gradient is maximized.

Orienting each of the at least two magnets can include varying the magnetization direction of each magnet in the magnet assembly until the time-average of the force metric is maximized or minimized over the region in which therapy is applied. For example, magnetization directions can depend on the radial distance from the magnet assembly and distance in from the front surface of the magnet assembly where the maximized the time-average of the force metric is desired, as described herein. Magnetization directions can also depend on the type of magnetically controllable objects to be conveyed. In some aspects, determining the magnetization direction of each magnet can include using a computing system operable to determine the optimal magnetization directions based on the type of magnetically controllable objects and the location where the time-average of the force metric is to be maximized or minimized.

In some aspects, the magnet assembly can have a first magnet, a second magnet, and/or a third magnet. The first magnet, second magnet, and/or third magnet can be fixed to one another and form a circular magnet. The first magnet, second magnet, and third magnet can each have a magnetization direction. The magnetization direction of the first magnet, second magnet, and third magnet can depend on a point to direct the magnetically controllable objects along the center axis of system, a radial distance from the center axis to provide a maximized time-averaged magnetic force, total magnet diameter (i.e., the diameter of the combined first magnet, second magnet, and third magnet when fixed to each other), the thickness of the magnet (i.e., the thickness of the first magnet, second magnet, and third magnet will be the same), and the type of magnetically controllable objects to be controlled. For example, the magnetically controllable objects can be magnetically saturated magnetic objects. In this example, for a point about 13 cm from the front surface of the magnet assembly along the center axis, a radial distance of about 11 cm from the center axis, a diameter of the combined magnet of about 18 cm, and a magnet thickness of about 6 cm, the first magnet magnetization direction can be about 151 degrees from the center axis, the second magnet magnetization direction can be about 90 degrees from the center axis, and the third magnet magnetization direction can be about 29 degrees from the center axis.

In another aspect, for magnetically saturated magnetic objects the point can be about 5 cm to about 20 cm from the front surface of the magnet assembly along the center axis, the radial distance can be about 5 cm to about 20 cm from the center axis, the diameter of the combined magnet can be about 5 cm to about 30 cm, and the magnet thickness can be about 2 cm to about 10 cm. In this example, the magnetization direction of the first magnet can be about 145 degrees to about 155 degrees from the center axis, or about 135 degrees to about 165 degrees from the center axis. The magnetization direction of the second magnet can be about 85 degrees to about 95 degrees from the center axis, or about 75 degrees to about 105 degrees from the center axis. The magnetization direction of the third magnet can be about 25 degrees to about 35 degrees from the center axis, or about 15 degrees to about 45 degrees from the center axis.

In another aspect, the magnetically controllable objects can be magnetically unsaturated magnetic objects. In this example, for a point about 13 cm from the front surface of the magnet assembly along the center axis, a radial distance of about 11 cm from the center axis, a diameter of the combined magnet of about 18 cm, and a magnet thickness of about 6 cm, the first magnet magnetization direction can be about 142 degrees from the center axis, the second magnet magnetization direction can be about 90 degrees from the center axis, and the third magnet magnetization direction can be about 38 degrees from the center axis.

In another example, for magnetically unsaturated magnetic objects, the point can be about 5 cm to about 20 cm from the front surface of the magnet assembly along the center axis, the radial distance can be about 5 cm to about 20 cm from the center axis, the diameter of the combined magnet can be about 5 cm to about 30 cm, and the magnet thickness can be about 2 cm to about 10 cm. In this example, the magnetization direction of the first magnet can be about 135 degrees to about 145 degrees from the center axis, or about 125 degrees to about 155 degrees from the center axis. The magnetization direction of the second magnet can be about 85 degrees to about 95 degrees from the center axis, or about 75 degrees to about 105 degrees from the center axis. The magnetization direction of the third magnet can be about 35 degrees to about 45 degrees from the center axis, or about 25 degrees to about 55 degrees from the center axis.

In another aspect, the magnet assembly can include two magnets, a first magnet and a second magnet. The first magnet and the second magnet can be cylinders fixed to a yolk and separated by a support column. The cylinders can be configured to remain in a fixed position in relation to the yolk and only rotate with a center axis of rotation defined by a motor spindle. The magnetization direction of the first magnet and second magnet can depend on a point to direct the magnetically controllable objects along the center axis of system, a radial distance from the center axis, a separation distance between the center of the first magnet and the center of the second magnet, and the type of magnetically controllable objects to be controlled.

In an example, the magnetically controllable objects can be magnetically saturated magnetic objects. The point to direct magnetically controllable objects toward the center axis can be about 13 cm from the front surface of the magnet assembly, the radial distance can be about 2 cm from the center axis, and the separation distance between the center of the first magnet and the center of the second magnet can be about 7.5 cm. In this example, the magnetization direction of the first magnet can be about 156 degrees from the center axis and the magnetization direction of the second magnet can be about 24 degrees from the center axis. In another example, the point to direct magnetically controllable objects toward the center axis can be about 5 cm to about 20 cm from the front surface of the magnet assembly, the radial distance can be about 1 cm to about 10 cm from the center axis, and the separation distance between the center of the first magnet and the center of the second magnet can be about 5 cm to about 15 cm. In this example, the magnetization direction of the first magnet can be about 150 degrees to about 160 degrees from the center axis, or about 130 degrees to about 170 degrees from the center axis. The magnetization direction of the second magnet can be about 20 degrees to about 30 degrees from the center axis, or about 10 degrees to about 40 degrees from the center axis.

In another example, the magnetically controllable objects can be magnetically unsaturated magnetic objects. The point to direct magnetically controllable objects toward the center axis can be about 13 cm from the front surface of the magnet assembly, the radial distance can be about 2 cm from the center axis, and the separation distance between the center of the first magnet and the center of the second magnet can be about 7.5 cm. In this example, the magnetization direction of the first magnet can be about 143 degrees from the center axis and the magnetization direction of the second magnet can be about 37 degrees from the center axis. In another example, the point to direct magnetically controllable objects toward the center axis can be about 5 cm to about 20 cm from the front surface of the magnet assembly, the radial distance can be about 1 cm to about 10 cm from the center axis, and the separation distance between the center of the first magnet and the center of the second magnet can be about 5 cm to about 15 cm. The magnetization direction of the first magnet can be about 135 degrees to about 150 degrees from the center axis, or about 130 degrees to about 155 degrees from the center axis. The magnetization direction of the second magnet can be about 30 degrees to about 45 degrees from the center axis, or about 25 degrees to about 50 degrees from the center axis.

At block 3006, the method 3000 includes providing a plurality of magnetically controllable objects. Any of the magnetically controllable objects described herein can be used with the method. In some examples, providing the plurality of magnetically controllable objects can include introducing the magnetically controllable objects into a subject and/or applying the magnetically controllable objects on to a body surface of the subject. In other examples, providing the plurality of magnetically controllable objects can include providing the objects to a surface or an internal area of a material. For example, the magnetically controllable objects can be magnetic particulates, magnetic nanoparticles, magnetic beads, magnetic spheres, magnetic cylinders, magnetic cubes, magnetic fluids, magnetic therapeutics, iron oxide nanoparticles, and magnetic contrast agents. Magnetically controllable objects can include magnetic nanoparticles to which therapeutics are attached. Magnetically controllable objects can include magnetic nanoparticles used to adjunctively convey therapeutic agents by creating artificial fluidic currents within a vasculature of a patient. Therapeutic agents which can be attached to magnetic nanoparticles, or be adjunctively conveyed by magnetic nanoparticles, include, but are not limited to, therapeutic agents used to treat cancer or provide neuroprotection.

At block 3008, the method 3000 can include conveying the magnetically controllable objects in the general direction of desired magnetically controllable object conveyance using the magnet system. The magnet assembly can be placed near the magnetically controllable objects. For example, the magnet system can be placed about 1 cm to about 20 cm from the magnetically controllable objects to be conveyed. In some examples, this distance is defined as the radial direction (e.g., distance between the center axis of the magnet system and the magnetically controllable objects to be controlled). The motor can provide a torque to the motor spindle, thereby rotating the magnet assembly. The motor is operable to provide rotation to the magnet assembly at a constant or variable rate. In an example, the constant rate can be about 1 Hz to about 2 Hs, about 2 Hz to about 3 Hz, about 3 Hz to about 4 Hz, about 4 Hz to about 5 Hz, about 5 Hz to about 6 Hz, about 6 Hz to about 7 Hz, about 7 Hz to about 8 Hz, about 8 Hz to about 9 Hz, about 9 Hz to about 10 Hz, or more. The constant or variable rate can be reversed in time. Once the magnet system is placed near the magnetically controllable objects and the magnet assembly begins rotating, at least a portion of the magnetically controllable objects are conveyed by a time-average force in a general direction of desired travel toward a preferred point or region or away from a preferred point or region. After a period of time, the magnetically controllable objects all move in the general direction of desired travel toward the preferred point or region or away from the preferred point or region. The magnet assembly generates a changing magnetic field in time which thereby generates the time-averaged magnetic force.

In some aspects, the method 3000 can further include removing the magnetically controllable objects from the subject. Removing the magnetically controllable objects can include moving the magnetically controllable objects to an aspiration catheter. In another example, the magnetically controllable objects can be moved to a retrievable magnetic object. The magnetically controllable objects can stick to the retrievable magnetic object and thereby be removed from the subject.

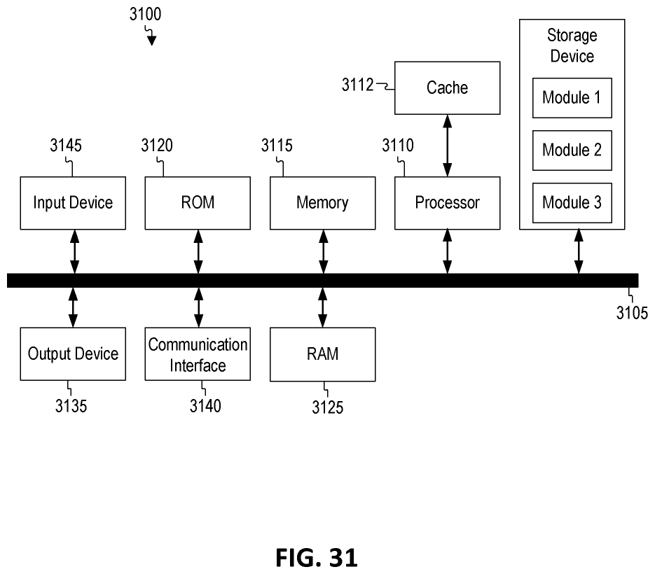

FIG. 31 is a diagram illustrating an example of a system for implementing certain aspects of the present technology (e.g., orienting the magnetization directions of the magnet system). In particular, FIG. 31 illustrates an example of computing system 3100, which can be for example any computing device making up an internal computing system, a remote computing system, a camera, or any component thereof in which the components of the system are in communication with each other using connection 3105. Connection 3105 can be a physical connection using a bus, or a direct connection into processor 3110, such as in a chipset architecture. Connection 2305 can also be a virtual connection, networked connection, or logical connection.

In some aspects, computing system 3100 is a distributed system in which the functions described in this disclosure can be distributed within a datacenter, multiple data centers, a peer network, etc. In some aspects, one or more of the described system components represents many such components each performing some or all of the function for which the component is described. In some aspects, the components can be physical or virtual devices.

Example computing system 3100 includes at least one processing unit (CPU or processor) 3110 and connection 3105 that couples various system components including system memory 3115, such as ROM 3120 and RAM 3125 to processor 3110. Computing system 3100 can include a cache 3112 of high-speed memory connected directly with, in close proximity to, or integrated as part of processor 3110.

Processor 3110 can include any general purpose processor and a hardware service or software service, such as services 3132, 3134, and 3136 stored in storage device 3130, configured to control processor 3110 as well as a special-purpose processor where software instructions are incorporated into the actual processor design. Processor 3110 may essentially be a completely self-contained computing system, containing multiple cores or processors, a bus, memory controller, cache, etc. A multi-core processor may be symmetric or asymmetric.

To enable user interaction, computing system 3100 includes an input device 3145, which can represent any number of input mechanisms, such as a microphone for speech, a touch-sensitive screen for gesture or graphical input, keyboard, mouse, motion input, speech, etc. Computing system 3100 can also include output device 3135, which can be one or more of a number of output mechanisms. In some instances, multimodal systems can enable a user to provide multiple types of input/output to communicate with computing system 3100. Computing system 3100 can include communications interface 3140, which can generally govern and manage the user input and system output. The communication interface may perform or facilitate receipt and/or transmission wired or wireless communications using wired and/or wireless transceivers, including those making use of an audio jack/plug, a microphone jack/plug, a universal serial bus (USB) port/plug, an Apple® Lightning® port/plug, an Ethernet port/plug, a fiber optic port/plug, a proprietary wired port/plug, a Bluetooth® wireless signal transfer, a BLE wireless signal transfer, an IBEACON® wireless signal transfer, an RFID wireless signal transfer, near-field communications (NFC) wireless signal transfer, dedicated short range communication (DSRC) wireless signal transfer, 802.11 WiFi wireless signal transfer, WLAN signal transfer, Visible Light Communication (VLC), Worldwide Interoperability for Microwave Access (WiMAX), IR communication wireless signal transfer, Public Switched Telephone Network (PSTN) signal transfer, Integrated Services Digital Network (ISDN) signal transfer, 3G/4G/5G/LTE cellular data network wireless signal transfer, ad-hoc network signal transfer, radio wave signal transfer, microwave signal transfer, infrared signal transfer, visible light signal transfer, ultraviolet light signal transfer, wireless signal transfer along the electromagnetic spectrum, or some combination thereof. The communications interface 3140 may also include one or more Global Navigation Satellite System (GNSS) receivers or transceivers that are used to determine a location of the computing system 3100 based on receipt of one or more signals from one or more satellites associated with one or more GNSS systems. GNSS systems include, but are not limited to, the US-based GPS, the Russia-based Global Navigation Satellite System (GLONASS), the China-based BeiDou Navigation Satellite System (BDS), and the Europe-based Galileo GNSS. There is no restriction on operating on any particular hardware arrangement, and therefore the basic features here may easily be substituted for improved hardware or firmware arrangements as they are developed.

Storage device 3130 can be a non-volatile and/or non-transitory and/or computer-readable memory device and can be a hard disk or other types of computer readable media which can store data that are accessible by a computer, such as magnetic cassettes, flash memory cards, solid state memory devices, digital versatile disks, cartridges, a floppy disk, a flexible disk, a hard disk, magnetic tape, a magnetic strip/stripe, any other magnetic storage medium, flash memory, memristor memory, any other solid-state memory, a compact disc read only memory (CD-ROM) optical disc, a rewritable compact disc (CD) optical disc, digital video disk (DVD) optical disc, a blu-ray disc (BDD) optical disc, a holographic optical disk, another optical medium, a secure digital (SD) card, a micro secure digital (microSD) card, a Memory Stick® card, a smartcard chip, a EMV chip, a subscriber identity module (SIM) card, a mini/micro/nano/pico SIM card, another integrated circuit (IC) chip/card, RAM, static RAM (SRAM), dynamic RAM (DRAM), ROM, programmable read-only memory (PROM), erasable programmable read-only memory (EPROM), electrically erasable programmable read-only memory (EEPROM), flash EPROM (FLASHEPROM), cache memory (L1/L2/L3/L4/L5/L #), resistive random-access memory (RRAM/ReRAM), phase change memory (PCM), spin transfer torque RAM (STT-RAM), another memory chip or cartridge, and/or a combination thereof.

The storage device 3130 can include software services, servers, services, etc., that when the code that defines such software is executed by the processor 2310, it causes the system to perform a function. In some aspects, a hardware service that performs a particular function can include the software component stored in a computer-readable medium in connection with the necessary hardware components, such as processor 3110, connection 3105, output device 3135, etc., to carry out the function. The term “computer-readable medium” includes, but is not limited to, portable or non-portable storage devices, optical storage devices, and various other mediums capable of storing, containing, or carrying instruction(s) and/or data. A computer-readable medium may include a non-transitory medium in which data can be stored and that does not include carrier waves and/or transitory electronic signals propagating wirelessly or over wired connections. Examples of a non-transitory medium may include, but are not limited to, a magnetic disk or tape, optical storage media such as CD or DVD, flash memory, memory or memory devices. A computer-readable medium may have stored thereon code and/or machine-executable instructions that may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, or the like.

In some cases, the computing device or apparatus may include various components, such as one or more input devices, one or more output devices, one or more processors, one or more microprocessors, one or more microcomputers, one or more cameras, one or more sensors, and/or other component(s) that are configured to carry out the steps of processes described herein. In some examples, the computing device may include a display, one or more network interfaces configured to communicate and/or receive the data, any combination thereof, and/or other component(s). The one or more network interfaces can be configured to communicate and/or receive wired and/or wireless data, including data according to the 3G, 4G, 5G, and/or other cellular standard, data according to the Wi-Fi (802.11x) standards, data according to the Bluetooth™ standard, data according to the IP standard, and/or other types of data.

The components of the computing device can be implemented in circuitry. For example, the components can include and/or can be implemented using electronic circuits or other electronic hardware, which can include one or more programmable electronic circuits (e.g., microprocessors, GPUs, DSPs, CPUs, and/or other suitable electronic circuits), and/or can include and/or be implemented using computer software, firmware, or any combination thereof, to perform the various operations described herein.

In some aspects the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

### EXAMPLES

A two magnet system was developed. The two magnet system is spun so that the temporal magnetic fields convey magnetic nanoparticles within the neurovasculature for the purpose of improving thrombolysis for acute ischemic stroke, where the thrombolytic agent is either adjunctively conveyed or conjugated to the magnetic nanoparticles.

The magnet system is relatively compact, measuring approximately 180 nm×180 nm×180 nm. A total magnet weight of 4.25 kg is employed to generate magnetic field aspects at a point 13.36 cm from the magnet system's origin (10.36 cm from the magnet system's front face). The magnetic field aspects are a 25 mT time averaged magnetic field, a −0.0029 T²/m time-averaged radial force metric for unsaturated magnetic nanoparticles where ρ=7.9 cm, and a −0.16 T/m time-averaged radial force metric for saturated magnetic nanoparticles where ρ=9.6 cm. ρ is the radial direction (distance) from a center axis (defined by the motor spindle) of the magnet system to the magnetically controllable objects.

Comparisons between theoretical and measured magnetic fields for the manufactured magnet show close agreement (measured magnetic field less than 5% or lower). The magnet system's maximum five-Gauss extent is 0.53 m. Operation frequencies are less than 5 Hz. The area over which magnetic nanoparticles are temporally conveyed towards the axis of rotation is broad (7.9 cm radius at 13.36 cm from the magnet's origin for magnetically unsaturated magnetic nanoparticles, 9.6 cm radius at 13.36 cm from the magnet's origin for magnetically saturated magnetic nanoparticles) which reduces the impact on efficacy with respect to precise magnet system placement.

The magnet system integrates two permanent magnet cylinders (6 cm diameter, 10 cm length, 322.6 A-m² for each), which are optimally oriented so that when the magnet assembly is spun about the central rotation axis (up to about 5 Hz), magnetic nanoparticles are preferentially conveyed within neurovascular vessels. The system is deployed after acute ischemic stroke (AIS) is confirmed, which includes the precise infusion of magnetic nanoparticles. The magnet system is small enough and low enough in weight such that it can travel with transferred patients (either within a hospital environment or via EMS transfers between hospitals). Procedures are typically about 15 minutes, but the magnet system can remain active longer such that necessary patient care is not withheld. The location of the clot can be identified from baseline CTA/MRA imaging prior to system activation, which enables the magnet system to be placed near the location of the suspected clot to achieve maximum effect. Because the temporal magnetic fields are capable of preferentially conveying magnetic nanoparticles over a broad region, precise knowledge of the clot's location is not necessary. However, conveyance efficacy can be improved if the clot's location is known.

The two magnets are optimally configured to maximize the temporal magnetic force (i.e., proportional to the force metric (∥β|∂B/∂ρ)) exerted upon unsaturated magnetic nanoparticles so that the magnetic nanoparticles are tumbled towards the magnet system's central axis of rotation). Theory predicts that for the two magnetic cylinders employed, magnetization angles of 33.91 and 146.09 degrees (with respect to the forward axis of rotation) with a magnet center-to-center separation distance of 8.5 cm will generate time-averages of 25 mT at a point 13.36 cm from the magnet system's origin (10.36 cm from the magnet system's front face). The strongest temporal force directed towards the rotational axis occurs at a radial distance of 7.9 cm for magnetically unsaturated magnetic nanoparticles (force metric of ∥B|∂B/∂ρ=0.0029 T²/m) and a radial distance of 9.6 cm for magnetically saturated magnetic nanoparticles (force metric of ∂B/∂ρ=−0.16 T/m).

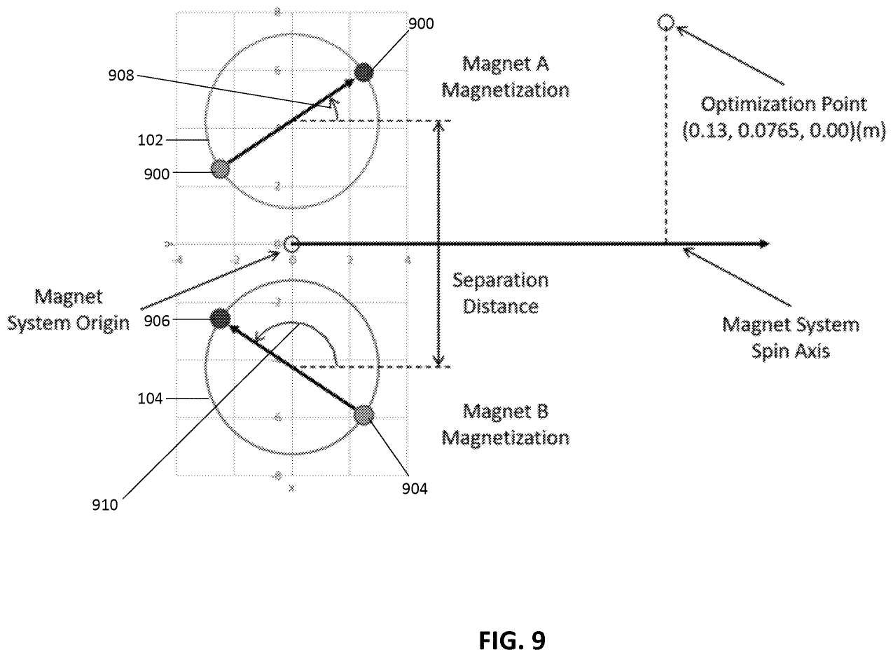

FIG. 9 illustrates the magnet design parameters. Although there are other magnet separations and magnetic cylinder alignment angles that improve upon the magnet criteria shown in FIG. 9, these separations and alignment angles result in stronger magnet-to-magnet forces and torques that substantially complicate magnet system assembly and operation. The angles and separation of the magnets were optimized so that the attractive force between magnet cylinders was less than about 530 N (about 120 pounds) and the magnetic torque induced between magnet cylinders was less than 4.6 N-m (3.4 ft-lbs). For this experiment, the magnet system was configured for use with magnetically unsaturated magnetic particles, however, the magnets, their separation, and angles can be reconfigured to maximize the time-average force applied to magnetically saturated nanoparticles (i.e., maximizing ∂B/∂ρ).

As illustrated in FIG. 9, the first magnet 102 has a south pole 900 and a north pole 902. The second magnet 104 has a north pole 906 and a south pole 904. The separation distance between the centers of the first magnet and the second magnet is about 8.5 cm. Angle 908 illustrates the magnetization angle (i.e., direction) of the first magnet 102. Angle 910 illustrates the magnetization angle (i.e., direction) of the second magnet 104. The optimization point illustrates where the strongest time-average magnetic force is exerted on magnetically unsaturated magnetic objects.

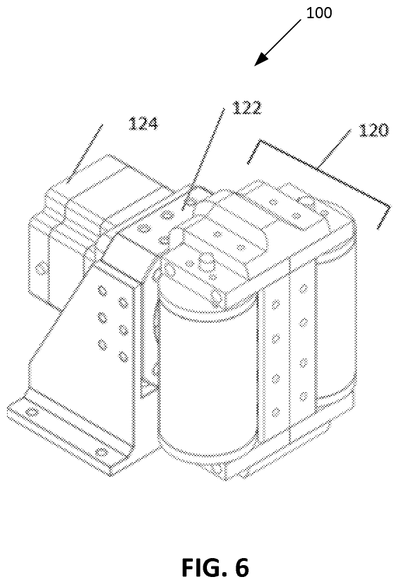

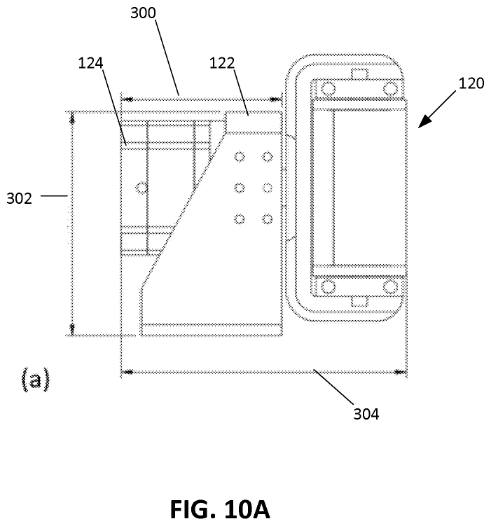

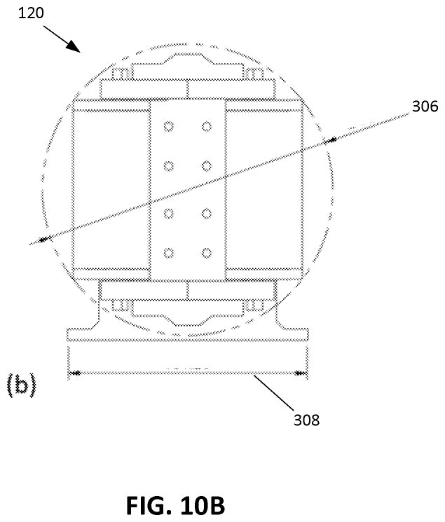

As illustrated in FIGS. 6 and 10A-10B, the magnet system possesses a small form factor which is need for portability. The magnet system can further include a cover (not shown). The magnet system 100 can have a total length 304 of about 183.2 mm. The motor 124 and base 122 assembly can have a length 300 of about 103.1 mm. The motor 124 and base 122 can have a height 302 of about 143.47 mm. The magnet assembly 120 can have a diameter 306 of about 183.83 and a total width 308 of about 151.25 mm.

To compute the magnetic fields generated by the magnet, a model was used which approximates the magnet as a collection of individual magnetic dipoles. Assuming high coercivity, the magnetization of each block corresponds to the permanent magnet's remnant magnetization. Each of the two magnetic cylinders is modeled as 256 individual magnetic blocks with a dipole magnetic field. For this, a remnant magnetization of 1.435 T+/−0.035T is used for Neodymium Boron Iron (NdBFe) magnet material (H_{c,b}=8.20e5 A/m). The density of NdBFe is 7.55 g/cm³. Table 1 illustrates the mechanical properties of the engineered magnet system.

TABLE 1
Magnet System Mechanical Properties

| Parameter | Value | Notes |
|---|---|---|
| Magnetization direction of | 33.910 | |
| first magnet with respect to | | |
| center axis (deg) | | |
| Magnetization direction of | 146.0890 | |
| second magnet with | | |
| respect to center axis | | |
| (deg) | | |
| Separation between | 8.5 | |
| magnet centers (cm) | | |
| x_optimized (m) | 0.13 | |
| y_optimized (m) | 0.0765 | |
| Z_optimized (m) | 0.00000 | |
| First Magnet Moment | 322.56 | |
| (A-m²) | | |
| First Magnet Weight (kg) | 2.1261 | |
| First Magnet Diameter (m) | 0.06 | |
| First Magnet Length (m) | 0.10 | |
| Second Magnet Moment | 322.56 | |
| (A-m²) | | |
| Second Magnet Weight | 2.1261 | |
| (kg) | | |
| Second Magnet Diameter | 0.06 | |
| (m) | | |
| Second Magnet Length | 0.10 | |
| (m) | | |
| First and Second Magnet | 4.2522 | |
| weight (kg) | | |
| First and Second Magnet | 359.9 | Along y-axis |
| Moment (A-m²) | | |
| COM (Center of Mass with | | |
| respect to the center axis | | |
| defined by the motor spindle) | | |
| x_com | 0 | |
| y_com | 0 | |
| z_com | 0 | |
| Mutual Force (N) | 530.5 | Along y-axis. Attractive |
| Mutual Torque (N-m) | 4.6042 | |
| Distance to cover face (m) | 0.03 | From COM |
| Magnet surface to cover | 0 | |
| (m) | | |
| x-distance to surface (mm) | 0.03 | |
| y-distance to surface (mm) | 0.920 | |
| z-distance to surface (mm) | 0.920 | |
| Neg. x-distance from | 0.03 | |
| surface (mm) | | |
| Grade | 52 MgOe - ShinEtsu | |
| NdBFe density (kg/m³) | 7550 | |
| Br-avg (T) | 1.4350 | |
| Br-error(T) | 0.0350 | |
| Br-error(+/−, fraction) | 0.244 | |

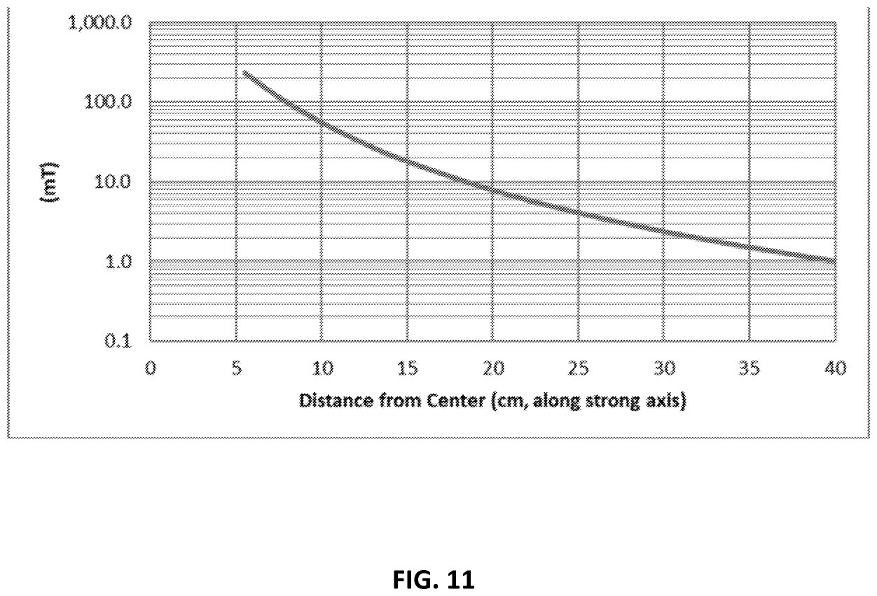

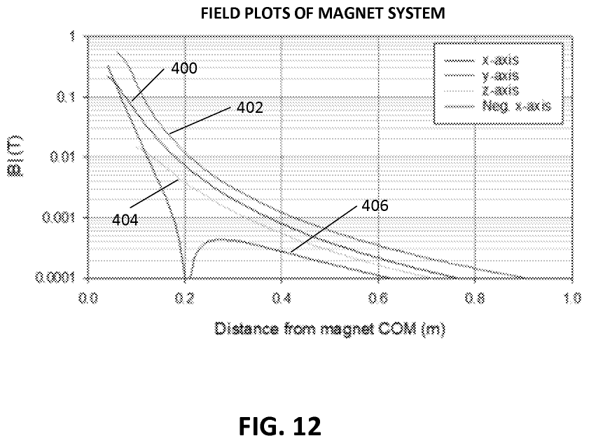

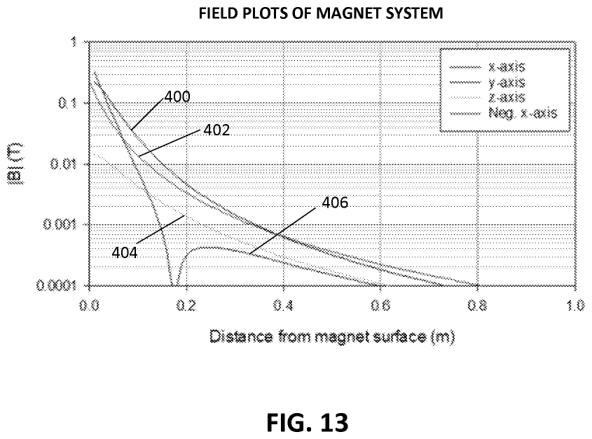

FIG. 11 illustrates the static magnetic field of a single Nd52 magnet cylinder. FIGS. 12 and 13 illustrate static magnetic fields of the magnet system along the cardinal axes showing x-axis 400, y-axis 402, z-axis 404, and negative x-axis 406. Magnetic field magnitudes are the same along +y and −y and along +z and −z axes due to magnet symmetry across x-z and x-y planes, respectively. Magnetic field magnitudes differ along +x and −x axes due to lack of magnet symmetry across the y-z plane.

Table 2 illustrates distances to five-Gauss (5G) along cardinal axes. This data corresponds to static magnetic field measurements. However, a sphere centered on the magnet system's origin with a radius of 53.3 cm fully encapsulated the 5G field for static and temporal magnetic fields.

TABLE 2
Five-Gauss (5G) distances along cardinal axes

| | |
|---|---|
| x-distance to 5G (m) | 0.4608 |
| y-distance to 5G (m) | 0.5324 |
| z-distance to 5G (m) | 0.4120 |
| Neg. x-distance to 5G (m) | 0.1866 |
| Max. 5G extent (m) | 0.5324 |

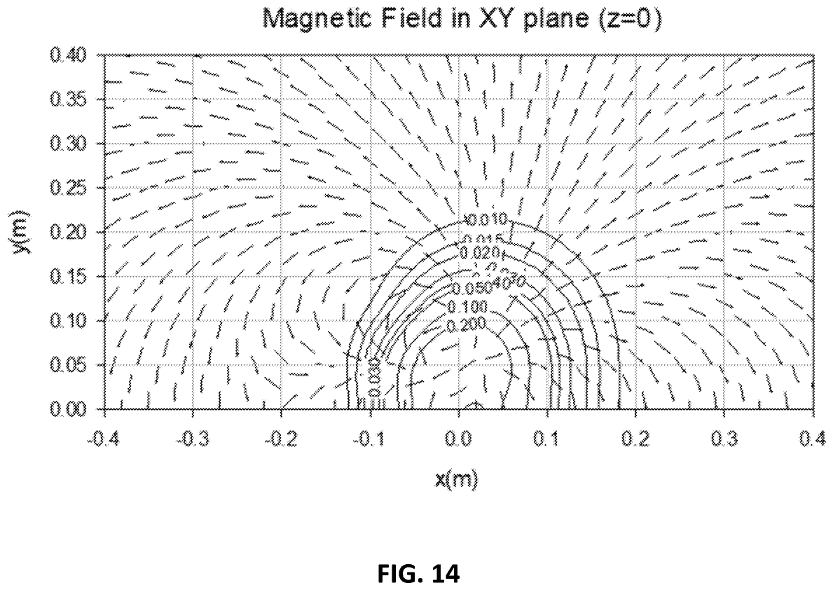

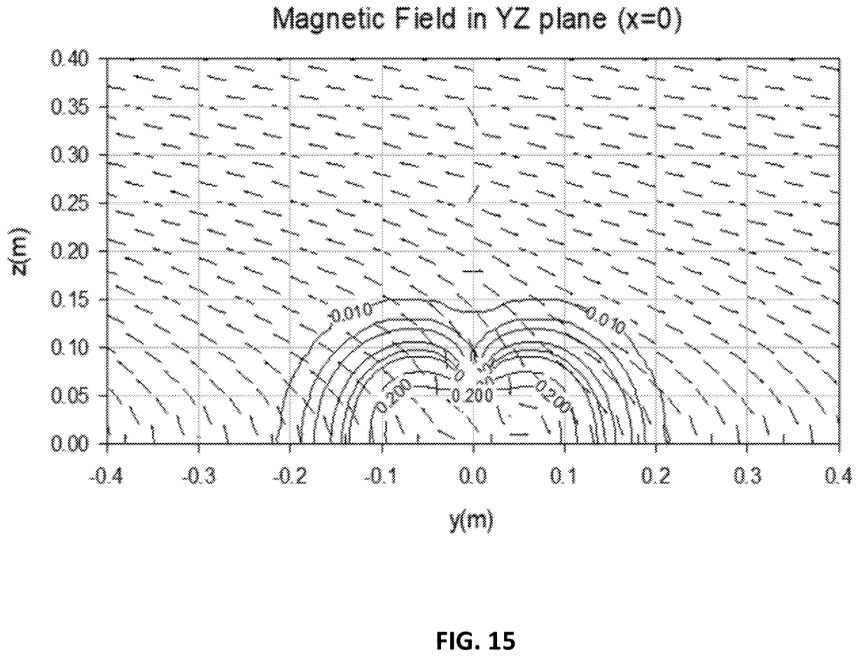

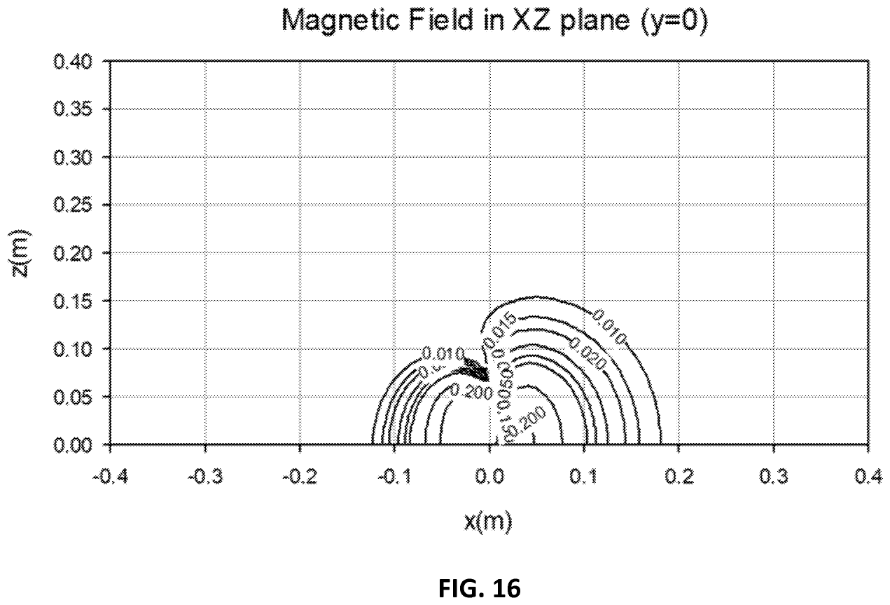

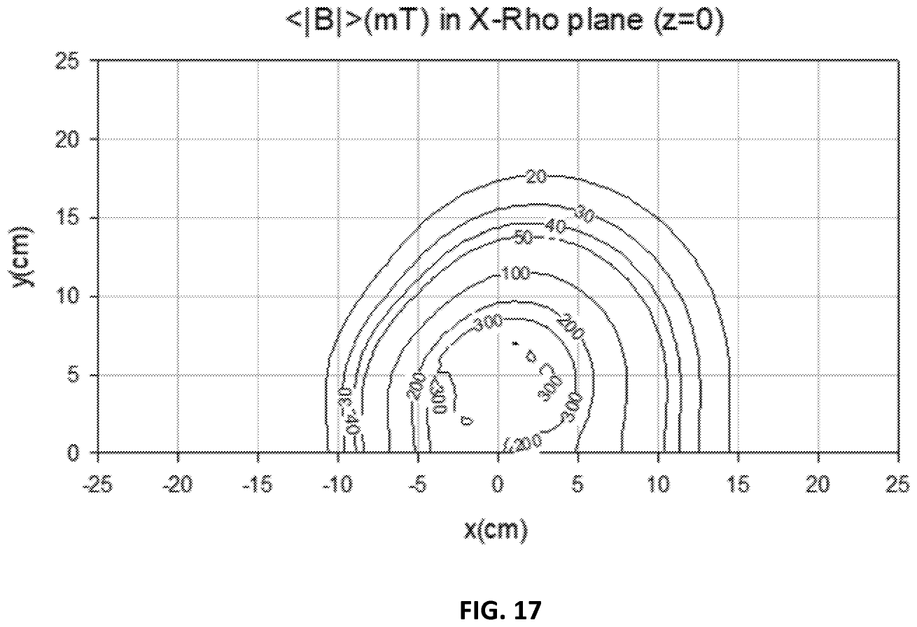

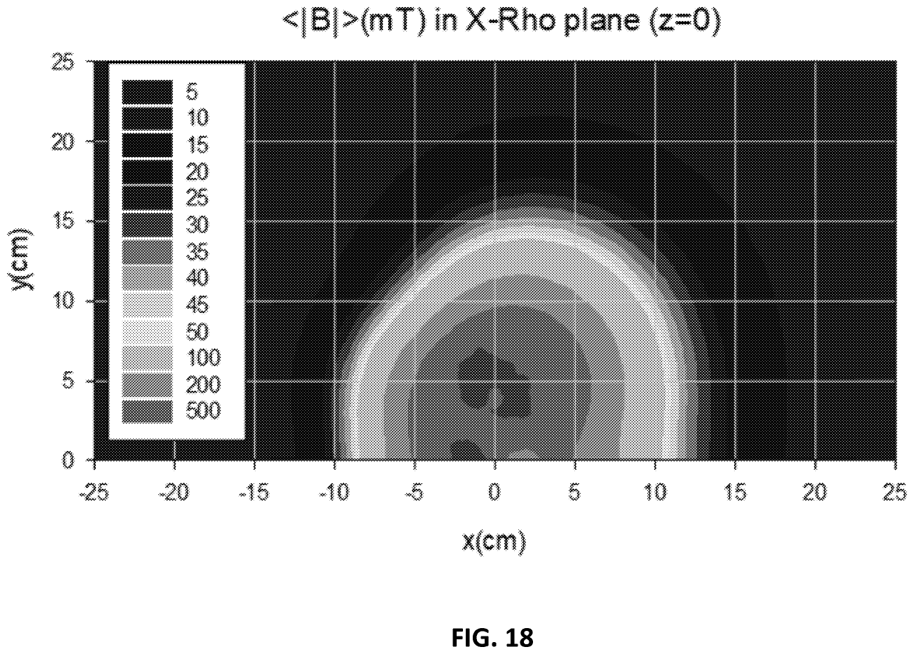

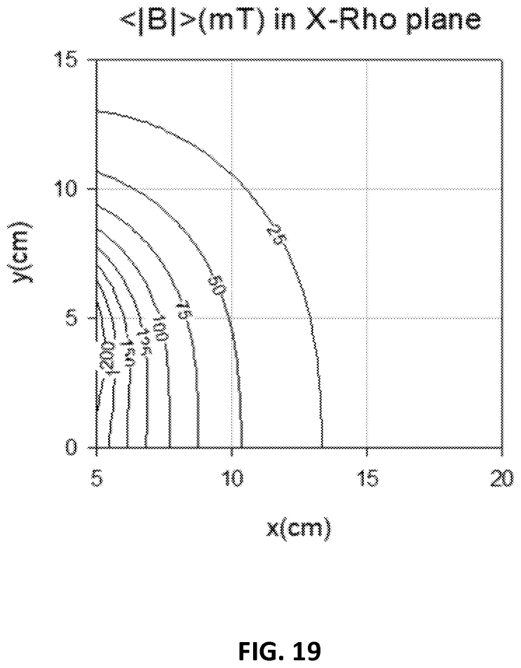

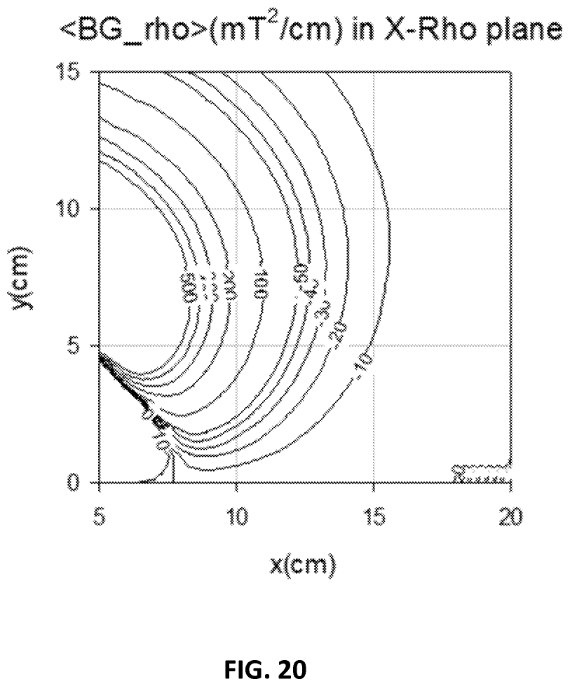

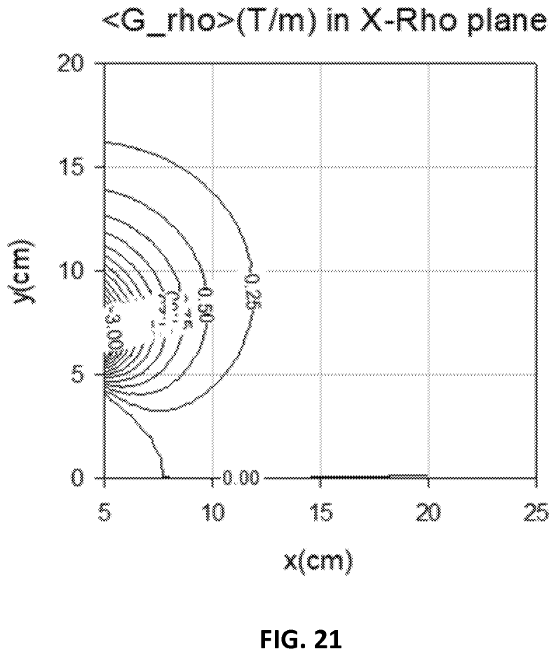

FIG. 14 illustrates a static magnetic field plot in the x-y plane (z=0). Contours show magnetic field magnitude (T). Vectors show directions of the magnetic field at points. FIG. 15 illustrates a static magnetic field plot in the y-z plane (x=0). Contours show magnetic field magnitude (T). Vectors show directions of the magnetic field at points. FIG. 16 illustrates a static magnetic field plot in the x-z plane (y=0). Contours show magnetic field magnitude (T). Vectors show directions of the magnetic field at points. All magnetic field directions point into the page. FIGS. 17-19 illustrates the time-average magnetic field generated by the magnet system in the x-y plane. FIG. 20 illustrates the time-average radial force metric in the x-y plane generated by the magnet system for magnetically unsaturated magnetic nanoparticles. FIG. 21 illustrates the time-average radial force metric in the x-y place generated by the magnet system for magnetically saturated magnetic nanoparticles.

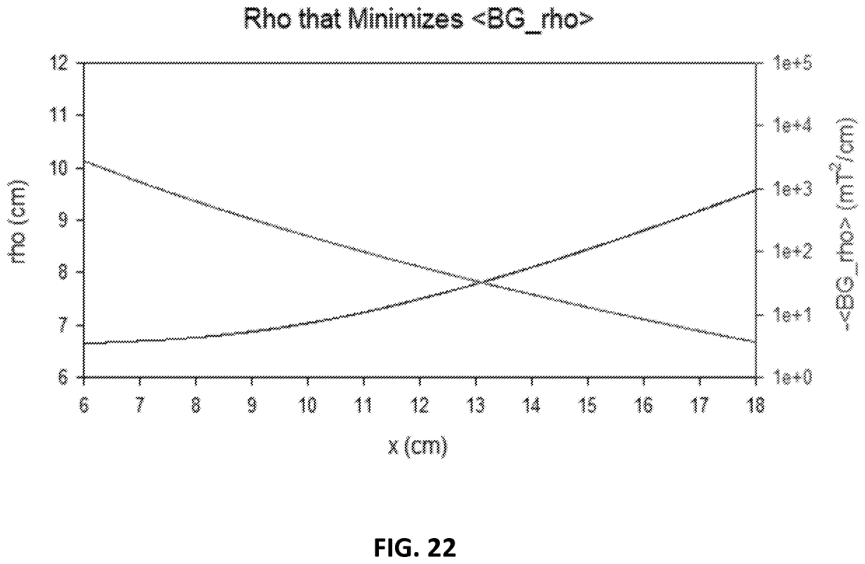

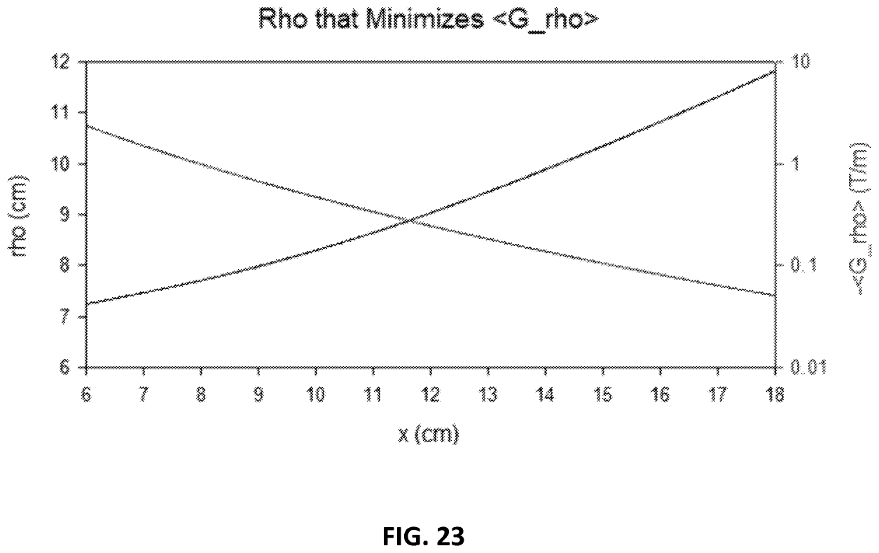

FIGS. 22-23 illustrate the radial location that maximizes the time-average radial force metric towards the magnet system's rotational axis. BG_rho denotes ∥β|∂B/∂ρ and G_rho denotes ∂B/∂p. Inward forces are negative in value, minimizing the radial force metric along the ρ axis is equivalent to maximizing the radial force metric towards the rotational axis (i.e., −ρ direction). Thus, minimizing BG_rho is equivalent to maximizing-BG_rho and minimizing G_rho is equivalent to maximizing −G_rho, where the negative sign denotes a direction opposite ρ (i.e., towards the axis of rotation).

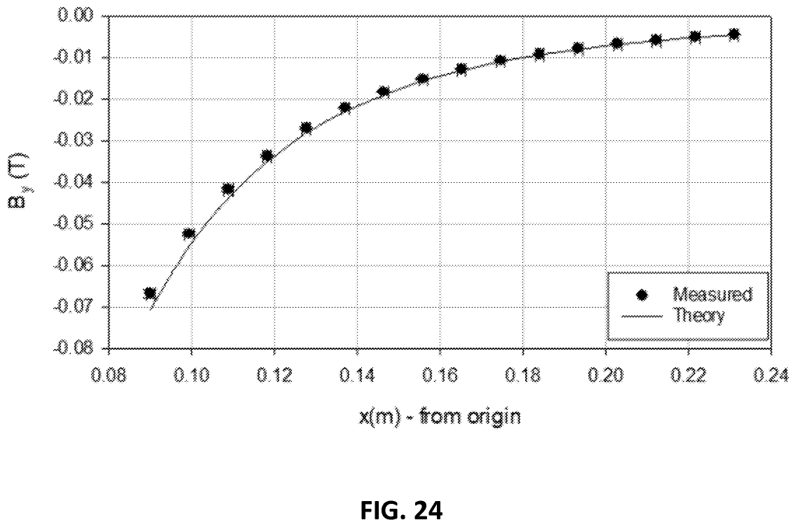

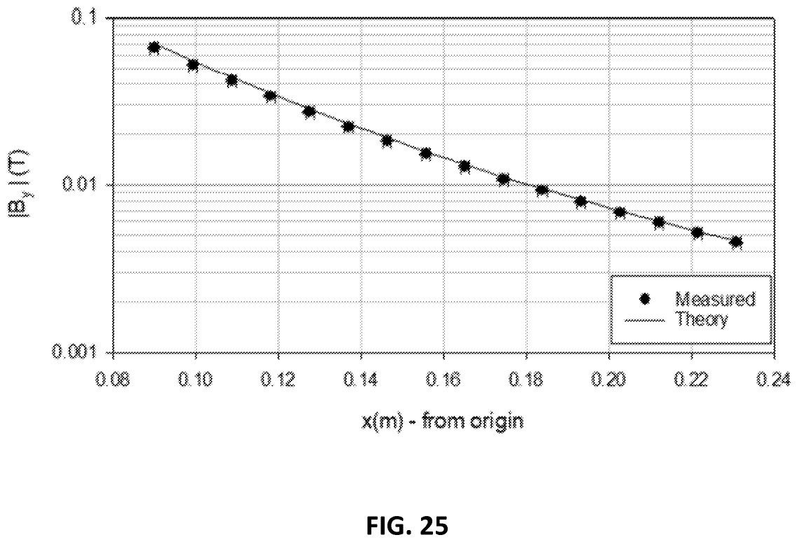

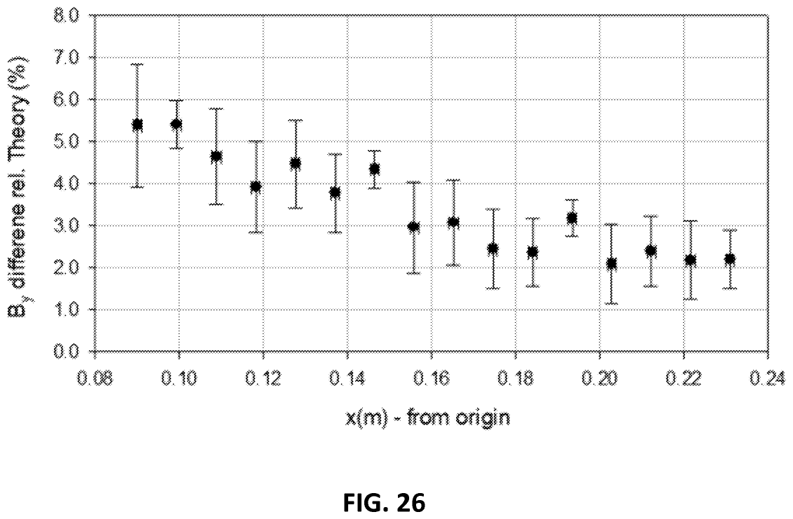

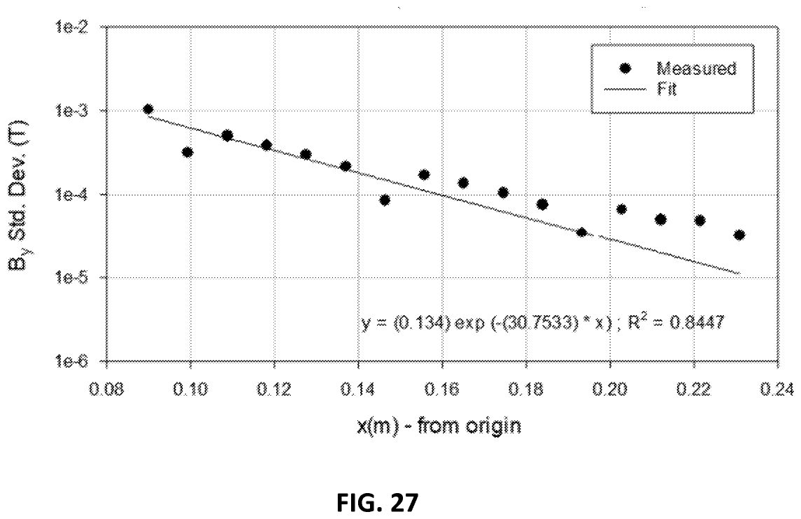

For comparison between theoretical magnetic fields to those generated by the magnet system, selected magnetic fields along the cardinal axes were compared. Five measurements of the magnetic field were made for each point using a multi-axis robotic arm, yielding adequate statistics to describe the actual magnetic fields generated by the system. It is estimated that position accuracy was about 1 mm (STD). The magnet system weighted 7.9 kg+/−0.05 kg (STD). FIGS. 24-26 illustrate static magnetic field comparison between theoretical and actual system measurements along the x-axis. The magnetic field is predicted to be strictly oriented in the y-direction along the x-axis. Thus, only the B_{y} magnetic field component is considered along the x-axis. The bidirectional error bars correspond to the standard deviation in magnetic field measurements and positional accuracy. FIG. 26 illustrates graph of the magnetic field differential between theoretical and measured magnetic fields generated by the magnetic system. FIG. 27 illustrates the change in standard deviation for static B_{y} measurements along the x-axis.

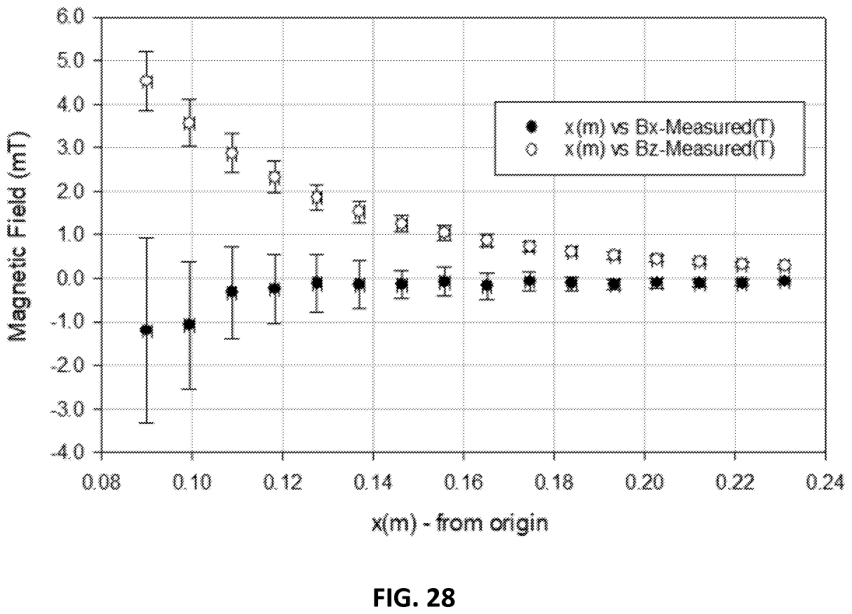

FIG. 28 illustrates the deviation of manufactured magnet's static magnetic field from the y-direction along the x-axis. The bidirectional error bars correspond to the standard deviation in magnetic field measurements and positional accuracy. The data suggest that the sampled points may not align with the magnet's rotational axis.

It was found that the magnet system performed close to the predictions of the theoretical model (within about 5%). It is possible that measured points along the x-axis did not fully align with the true axis of rotation. Possible axial misalignment accounts for a few percent deviation in the magnetic field measurements. It is also possible that the ShinEtsu Nd52 material was at the lower end of the material's remnant magnetization specification (1.40 T≤Bᵣ≤1.47 T) compared to what was modeled (1.435 T). The impact of a lower remnant magnetization would be a reduction of theoretical magnetic fields by 2.5%. Taken together, these two errors may account for the small magnetic field differences observed between theory and measured data. However, a limitation of the present theoretical model is that internal demagnetization forces are not reflected (a known aspect of permanent magnet materials). While this effect is negligible for permanent magnet materials with very high coercivities, it is possible that the ShinEtsu Nd52 material may suffer from some small degree of demagnetization given the moderate magnetic coercivity of this material.

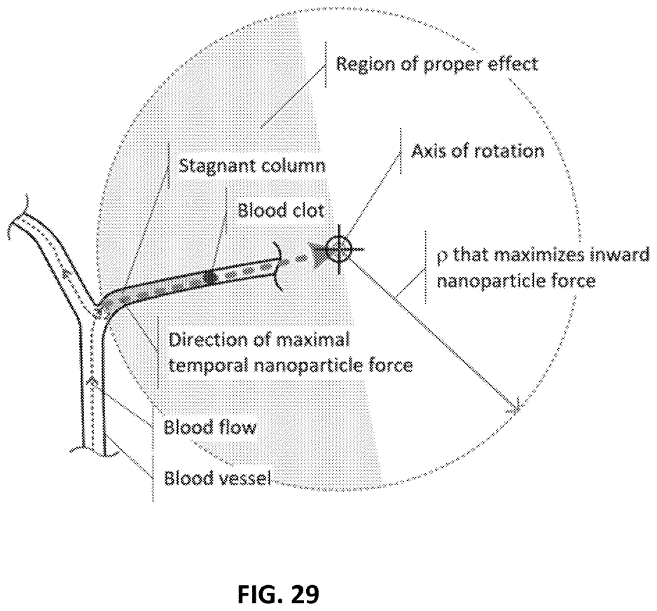

FIG. 29 illustrates magnet placement for maximal effect. With respect to best magnet placement to ensure the magnetic nanoparticles are conveyed towards the rotation axis, analysis shows that the magnet should be placed so that the nanoparticle capture point within flow (i.e., at the start of the stagnant column) aligns with the radial position (p) so that temporal magnetic forces on the magnetic nanoparticles are maximized towards the rotational axis. In addition, the navigational target (i.e., the clot) should be along the line from the optimal radial position to the rotational axis, as illustrated in FIG. 29, with the rotational axis placed distal to the target. Inward radial nanoparticle forces will also be generated over a semicircular region, albeit at lesser strengths.

The disclosures shown and described above are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the examples described above may be modified within the scope of the appended claims.

What is claimed is:

1. A system for controlling movement of one or more magnetically controllable objects, the system comprising:
a magnet assembly, the magnet assembly comprising at least two magnets, wherein each magnet of the at least two magnets has a magnetization direction;
a motor coupled to a motor spindle; and
a mounting mechanism configured to mount the magnet assembly to the motor spindle,
wherein the magnet assembly is configured to rotate along a center axis defined by the motor spindle.

2. The system of claim 1, wherein the at least two magnets comprise a first magnet, a second magnet, and a third magnet.

3. The system of claim 2, wherein the magnet assembly has a center of mass aligned with the center axis to ensure stable rotation about the center axis.

4. The system of claim 1, wherein the magnet assembly generates a changing magnetic field in time which induces at least a portion of the one or more magnetically controllable objects to move towards a preferred point or region in space by generating a time-averaged magnetic force in a general direction of desired travel towards the preferred point or region.

5. The system of claim 4, wherein after a period of time the one or more magnetically controllable objects all move in the general direction of desired travel towards the preferred point or region.

6. The system of claim 1, wherein the magnet assembly generates a changing magnetic field in time which induces at least a portion of the one or more magnetically controllable objects to move away from a preferred point or region in space by generating a time-averaged magnetic force in a general direction of desired travel away from the preferred point or region.

7. The system of claim 6, wherein after a period of time the one or more magnetically controllable objects all move in the general direction of desired travel away from the preferred point or region.

8. The system of claim 1, wherein the one or more magnetically controllable objects are permeable magnetic objects that are magnetically saturated or unsaturated, electrically conductive materials, permanent magnets, or any combination thereof.

9. The system of claim 8, wherein a time-averaged force in a general direction of desired magnetically controllable object conveyance for saturated magnetic objects is increased by orienting the magnetization directions of the at least two magnets in the magnet assembly such that a magnitude of a time average of a first order spatial gradient of a magnetic field in the general direction of desired magnetically controllable object conveyance is maximized.

10. The system of claim 8, wherein a time-averaged force in a general direction of desired magnetically controllable object conveyance for unsaturated magnetic objects is increased by orienting the magnetization directions of the at least two magnets in the magnet assembly such that a magnitude of a time average of a product of a magnetic field and a first order spatial gradient of the magnetic field in the general direction of desired magnetically controllable object conveyance is maximized.

11. The system of claim 1, wherein the system further comprises a cover configured to enclose the system.

12. The system of claim 11, wherein the system further comprises one or more attachment mechanisms for attaching the cover to the system.

13. The system of claim 1, wherein the at least two magnets comprise a distribution of permanent magnetic materials.

14. The system of claim 1, wherein the magnet assembly is rotated by the motor spindle at a constant rate or a variable rate.

15. The system of claim 14, wherein the constant rate or the variable rate is reversed in time.

16. The system of claim 1, wherein the one or more magnetically controllable objects are selected from the group consisting of magnetic particulates, magnetic nanoparticles, magnetic beads, magnetic spheres, magnetic cylinders, magnetic cubes, magnetic fluids, magnetic therapeutics, iron oxide nanoparticles, magnetic contrast agents, magnetic particles used to adjunctively convey therapeutic agents creating artificial fluidic currents within a vasculature, and magnetic nanoparticles having therapeutic agents attached.

17. The system of claim 1, wherein the magnet assembly is configured to only rotate about the center axis.

18. A method for conveying magnetically controllable objects, the method comprising:
providing a magnet system, the magnet system comprising:
a magnet assembly, the magnet assembly comprising at least two magnets, wherein each magnet of the at least two magnets has a magnetization direction;
a motor coupled to a motor spindle; and
a mounting mechanism configured to mount the magnet assembly to the motor spindle;
orienting the magnetization direction of each of the at least two magnets to increase a time-average force in a general direction of desired magnetically controllable object conveyance;
introducing a plurality of magnetically controllable objects into a subject; and
conveying the magnetically controllable objects in the general direction of desired magnetically controllable object conveyance using the magnet system.

19. The method of claim 18, wherein the time-average force is increased in saturated magnetic objects by orienting the magnetization directions of the at least two magnets such that a magnitude of a first order spatial gradient of a magnetic field is maximized.

20. The method of claim 18, wherein the time-average force is increased in unsaturated magnetic objects by orienting the magnetization directions of the at least two magnets such that a magnitude of a time average of a product of a magnetic field and a first order spatial gradient of the magnetic field is maximized.
